(19) United States
(12) Patent Application Publication
(10) Pub. No.: US 2026/0191175 A1
(43) Pub. Date: Jul. 9, 2026

(54) FISH INDIVIDUAL DEFICIENT IN AT LEAST PART OF FUNCTION OF STERILIZATION PROTEIN

(71) Applicant: REGIONAL FISH INSTITUTE, LTD., Kyoto (JP)

(72) Inventors: Kenta KISHIMOTO, Kyoto (JP); Ricardo Shohei HATTORI, Kyoto (JP); Atsushi KUNII, Kyoto (JP); Yuki HONDA, Kyoto (JP)

(73) Assignee: REGIONAL FISH INSTITUTE, LTD., Kyoto (JP)

(21) Appl. No.: 19/134,378

(22) PCT Filed: Aug. 9, 2024
(86) PCT No.: PCT/JP2024/028805

§ 371 Date: May 30, 2025

(30) Foreign Application Priority Data
Sep. 4, 2023 (JP) 2023-143263

(51) Int. Cl. A01K 67/0278; C07K 14/435

(52) U.S. Cl. CPC A01K 67/0278; C07K 14/43595; A01K 2217/052; A01K 2217/072; A01K 2227/40

(57) ABSTRACT

An object of the present invention is to provide a fish individual which, as shown in FIG. 2, makes it possible, in producing a germ-cell deficient individual, to identify a genotype of a sterilization protein by appearance without collection of a portion of a fish body. This object is achieved by a fish individual deficient in at least a part of a function of the sterilization protein, the fish individual having, in at least one of sterilization gene alleles encoding the sterilization protein which indicates sterility by at least the part of the function thereof being deficient, a polynucleotide sequence encoding the fluorescent protein to be expressible.

### TECHNICAL FIELD

The present invention relates to a fish individual deficient in at least a part of a function of a sterilization protein, a method for producing the fish individual, a method for transplanting a polynucleotide and a germ cell, and a method for identifying a genotype.

### BACKGROUND ART

The dead end (dnd) gene has been known as one of sterilization genes associated with sterilization. It has also been known that an individual deficient in a function of a protein encoded by the sterilization gene (hereinafter also referred to as a “sterilization protein”) is sterilized due to lack of germ cell formation.

### SUMMARY OF INVENTION

### Technical Problem

In a conventional method for artificially producing a germ-cell deficient individual deficient in a germ cell by taking advantage of the deficiency in the function of the sterilization protein, as shown in FIG. 1, hetero types heterozygously deficient in the function of the sterilization protein are crossed with each other to produce a homo type homozygously deficient in a function of a sterilization gene. The next generation produced by crossing the hetero types with each other can be a wild type, homo type, or hetero type. However, it is impossible to identify these genotypes by appearance. Therefore, in order to identify the genotype, it is necessary to collect a portion of a fish body and perform a genetic analysis.

An object of the present invention is to provide a fish individual which, as shown in FIG. 2, makes it possible, in producing a germ-cell deficient individual, to identify a genotype of a sterilization protein by appearance without collection of a portion of a fish body.

### Solution to Problem

### Item 1.

A fish individual deficient in at least a part of a function of a sterilization protein, the fish individual having, in at least one of sterilization gene alleles encoding the sterilization protein which indicates sterility by at least the part of the function thereof being deficient, a polynucleotide sequence encoding a fluorescent protein to be expressible.

### Item 2.

The fish individual according to Item 1 having the polynucleotide sequence encoding the fluorescent protein in both of the sterilization gene alleles and being deficient in reproductive capacity.

### Item 3.

The fish individual according to Item 2 having polynucleotide sequences encoding the fluorescent protein and having the same fluorescent wavelength band in both of the sterilization gene alleles.

### Item 4.

The fish individual according to Item 2 having a polynucleotide sequence encoding the fluorescent protein and having the different fluorescent wavelength band in each of the sterilization gene alleles.

### Item 5.

The fish individual according to Item 1, in which the sterilization gene is a dead end (dnd) gene.

### Item 6.

The fish individual according to Item 1, in which an expression site of the fluorescent protein is an eye.

### Item 7.

A production method of a fish individual deficient in at least a part of a function of a sterilization protein, which indicates sterility by at least the part of the function thereof being deficient, the method including:
transfecting a polynucleotide sequence encoding the fluorescent protein to be expressible in at least one of sterilization gene alleles encoding the sterilization protein.

### Item 8.

The production method according to Item 7, in which the transfection is performed by a genome editing method or a knock-in method using a gene site-specific recombination method.

### Item 9.

The production method according to Item 8, in which the genome editing method is a method using at least one system selected from a clustered regularly interspaced short palindromic repeats/CRISPR associated protein 9 (CRISPR/Cas9) system, CompoZr zinc finger nuclease (ZFN) system, and a TAL effector nuclease (TALEN) system.

### Item 10.

The production method according to Item 8, in which the gene site-specific recombination method is a method using at least one system selected from a Cre/loxP system, a Flp/FRT system, a Dre/rox system, and a PhiC31 integrase system.

### Item 11.

A polynucleotide including: a polynucleotide sequence encoding a fluorescent protein to be expressible in a part of a sequence of a sterilization gene encoding a sterilization protein, which indicates sterility by at least a part of a function thereof being deficient.

### Item 12.

A method for transplanting a germ cell, the method including: transplanting a germ cell of a different fish species from the fish individual according to Item 2 into the fish individual.

### Item 13.

A method for identifying a genotype, the method including: mating male and female fish individuals, each of which has a polynucleotide sequence encoding a fluorescent protein to be expressible only in one of sterilization gene alleles of the fish individual according to claim 1; and observing expression of the fluorescent protein in an embryonic body or adult fish produced by mating.

### Advantageous Effects of Invention

The present invention can provide the fish individual which makes it possible, in producing the germ-cell deficient individual, to identify the genotype of the sterilization protein by appearance.

### BRIEF DESCRIPTION OF DRAWINGS

FIG. 1 shows a conventional method.

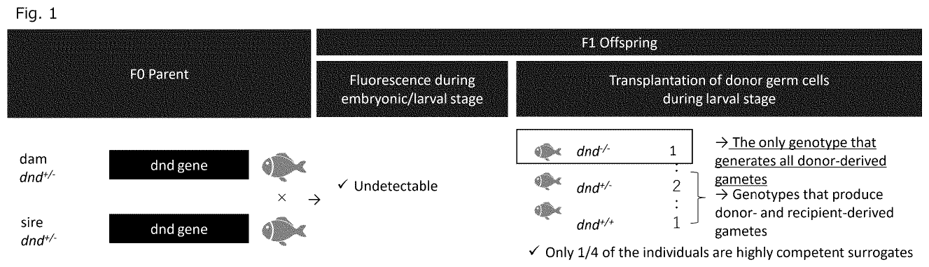

FIG. 2 shows an outline of the present invention.

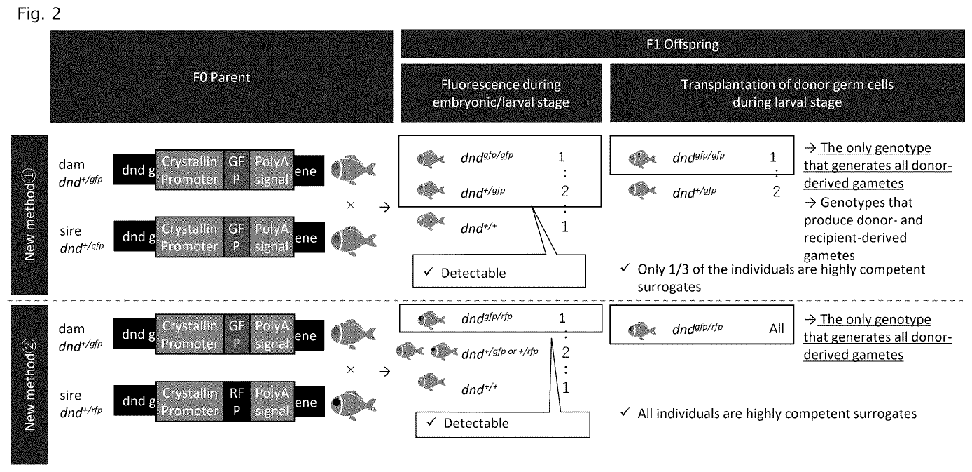

FIG. 3 shows a transgene map for a tilapia.

FIG. 4 shows fertilized eggs, embryoid bodies, and expression of fluorescent proteins in adult tilapia eyes.

FIG. 5 shows a position of a primer designed to confirm correct transfection of a cassette polynucleotide into a tilapia dnd gene.

FIG. 6 shows results of PCR using the primer shown in FIG. 5.

FIG. 7 shows sequences at cassette polynucleotide transfection sites in a dnd^{gfp} individual of the tilapia. (A) shows a 5′-side sequence analyzed by using an Onil_dnd_5-prime_Fw primer. (B) shows a 3′-side sequence analyzed by using an Onil_dnd_3-prime_Rv primer.

FIG. 8 shows sequences at cassette polynucleotide transfection sites in a dnd^{rfp} individual of the tilapia. (A) shows a 5′-side sequence analyzed by using the Onil_dnd_5-prime_Fw primer. (B) shows a 3′-side sequence analyzed by using the Onil_dnd_3-prime_Rv primer.

FIG. 9 shows a transgene map for a grass puffer.

FIG. 10 shows fertilized eggs, embryoid bodies, and expression of fluorescent proteins in adult grass puffer eyes.

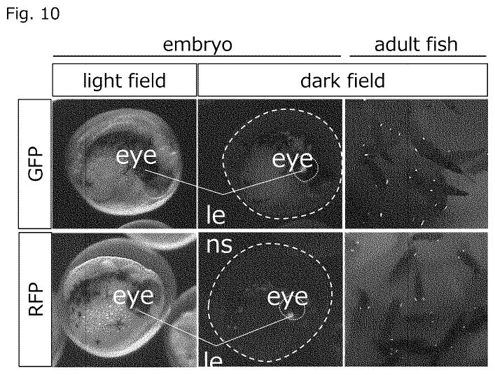

FIG. 11 shows a position of the primer designed to confirm the correct transfection of the cassette polynucleotide into a grass puffer dnd gene.

FIG. 12 shows results of the PCR using the primer shown in FIG. 11.

FIG. 13 shows sequences at cassette polynucleotide transfection sites in a dnd^{gfp} individual of the grass puffer. (A) shows the 5′-side sequence analyzed by using the Talb_dnd_5-prime_Fw primer. (B) shows the 3′-side sequence analyzed by using the Talb_dnd_3-prime_Rv primer.

FIG. 14 shows sequences at cassette polynucleotide transfection sites in a dnd^{rfp} individual of the grass puffer. (A) shows the 5′-side sequence analyzed by using the Talb_dnd_5-prime_Fw primer. (B) shows the 3′-side sequence analyzed by using the Talb_dnd_3-prime_Rv primer.

### DESCRIPTION OF EMBODIMENTS

### 1. Fish Individual Deficient in at Least Part of Function of Sterilization Protein

An embodiment relates to a fish individual deficient in at least a part of a function of a sterilization protein which indicates sterility due to deficiency in at least a part of a function thereof (hereinafter also simply referred to as a “fish individual”). The fish individual has a polynucleotide sequence encoding a fluorescent protein to be expressible in at least one of sterilization gene alleles encoding the sterilization protein.

The “fish individuals deficient in at least the part of the function of the sterilization protein” can include, as genotypes: a hetero type in which the polynucleotide sequence encoding the fluorescent protein is present in only one of the alleles in the fish individual; and a homo type in which the polynucleotide sequence encoding the fluorescent protein is present in both of the alleles.

In the hetero type, transfection of the polynucleotide sequence encoding the fluorescent protein into one of the sterilization gene alleles results in deficiency in the function of the sterilization protein encoded in the respective allele. The hetero type is phenotypically an individual that retains at least some of the germ cells (also referred to as a “hetero-deficient individual” in the present description), and both sexes thereof are reproductive.

In the homo type, the transfection of the polynucleotide sequence encoding the fluorescent protein into both of the sterilization gene alleles results in deficiency in the functions of the sterilization proteins in both alleles. The homo type is phenotypically an individual that is substantially deficient in the germ cell (also referred to as a “homo-deficient individual” in the present description), and both sexes thereof are deficient in reproductive capacity. The deficiency in the germ cell can be confirmed by histological observation. For example, in a case where the number of germ cells (primordial germ cells, spermatogonia, oogonia, spermatocytes, oocytes, spermatids, ootids, ova, or sperms) is reduced to 50% or less, 30% or less, 20% or less, 10% or less, 5% or less, 3% or less, 1% or less than that of a wild-type individual, the deficiency in the germ cells can be determined.

The term “fish” is not particularly limited.

In the present description, the fish can include saltwater fish, freshwater fish, brackish water fish, diadromous fish, and the like.

Examples of the fish include fishes of the families Paralichthys, Tetraodontidae: puffers, Ostraciidae: boxfishes, Sparidae: sea breams and porgies, Salmonidae, Cyprinidae, Ictaluroidea, Siluroidea, Bagroidea, Serranidae: sea basses, Cichlidae, Oryziidae: medakas, Monacanthidae, Osmeridae, Scombridae, Pleuronectidae, Carangidae, Lateolabrax, Moronidae, Latidae, Rachycentridae, Cynoglossidae, Anguillidae, Congridae, and the like.

Fishes of the family Paralichthys can include, for example, Paralichthys olivaceus (TEMMINCK et SCHLEGEL), fishes belonging to the genus Pseudorhombus (ocellated flounder (Pseudorhombus dupliciocellatus), roughscale flounder (Pseudorhombus oligodon), Pseudorhombus ctenosquamis, largetooth flounder (Pseudorhombus arsius), fivespot flounder (Pseudorhombus pentophthalmus), cinnamon flounder (Pseudorhombus cinnamoneus), central spotted flounder (Pseudorhombus levisquamis), and the like), fishes belonging to the genus Tarphops (small flounder (Tarphops oligolepis) and the like), fishes belonging to the genus Asterorhombus (intermediate flounder (Asterorhombus intermedius)), fishes belonging to the genus Lepidopsetta (dusky sole (Lepidopsetta mochigarei)), fishes belonging to the genus Taeniopsetta (Indo-Pacific ocellated flounder (Taeniopsetta ocellata)), and the like. A preferred fish of the family Paralichthys is Paralichthys olivaceus (TEMMINCK et SCHLEGEL).

Fishes of the family Tetraodontidae can include, for example: fishes belonging to the genus Takifugu such as torafugu (Takifugu rubripes), purple puffer (Takifugu porphyreus), and grass puffer (Takifugu niphobles); fishes belonging to the genus Lagocephalus such as half-smooth golden pufferfish (Lagocephalus wheeleri); and the like. Fishes of the family Ostraciidae can include, for example, fishes belonging to the genus Ostracion such as bluespotted boxfish (Ostracion immaculatus).

Fishes of the family Sparidae can include, for example: fishes belonging to the genus Pagrus such as red seabream (Pagrus major) and squirefish (Pagrus auratus); fishes belonging to the genus Acanthopagrus such as black porgy (Acanthopagrus schlegelii) and yellowfin seabream (Acanthopagrus latus); fishes belonging to the genus Dentex such as yellowback seabream (Dentex tumifrons); fishes belonging to the genus Sparus such as gilthead seabream (Sparus aurata); and the like.

Fishes of the family Salmonidae can include, for example: fishes belonging to the genus Oncorhynchus such as rainbow trout (Oncorhynchus mykiss), Chinook salmon (Oncorhynchus tshawytscha), cherry salmon (Oncorhynchus masou masou), red-spotted masu salmon (Oncorhynchus masou ishikawae), black kokanee (Oncorhynchus kawamurae), pink salmon (Oncorhynchus gorbuscha), and chum salmon (Oncorhynchus keta); fishes belonging to the genus Salmo such as brown trout (Salmo trutta), sockeye salmon (Oncorhynchus nerka), coho salmon (Oncorhynchus kisutch), and Atlantic salmon (Salmo salar); fishes belonging to the genus Salvelinus such as Dolly Varden (Salvelinus malma), Arctic char (Salvelinus alpinus), whitespotted char (Salvelinus leucomaenis), brook trout (Salvelinus fontinalis), and lake trout (Salvelinus namaycush); fishes belonging to the genus Hucho such as Sakhalin taimen (Parahucho perryi); and the like.

Fishes of the family Cyprinidae can include, for example, Honmoroko (Gnathopogon caerulescens), silver carp (Hypophthalmichthys molitrix), Eurasian carp (Cyprinus carpi), grass carp (Ctenopharyngodon idellus), bighead carp (Hypophthalmichthys nobilis), crucian carp (Carassius carassius), catla (Cyprinus catla), black carp (Mylopharyngodon piceus), mud carp (Cirrhinus molitorella), mrigal carp (Cirrhinus cirrhosus), catla (Catla catla), rohu (Labeo rohita), Wuchang bream (Megalobrama amblycephala), and the like.

Fishes of the superfamily Ictaluroidea can include, for example, American catfish (Ictalurus punctatus), blue catfish (Ictalurus furcatus), and the like.

Fishes of the superfamily Siluroidea can include, for example, Amur catfish (Silurus asotus), Lake Biwa catfish (Silurus biwaensis), rock catfish (Silurus lithophilus), Wels catfish (Silurus glanis), whitespotted clarias (Clarias fuscus), walking catfish (Clarias batrachus), and the like.

Fishes of the superfamily Bagroidea can include, for example, yellow catfish (Pseudobagrus fulvidraco), Mekong giant catfish (Pangasianodon gigas), basa (Pangasius bocourti), striped catfish (Pangasianodon hypophthalmus), and the likes.

Fishes of the family Serranidae can include, for example: fishes belonging to the genus Epinephelus such as convict grouper (Epinephelus septemfasciatus), longtooth grouper (Epinephelus bruneus), Hong Kong grouper (Epinephelus akaara), Malabar grouper (Epinephelus malabaricus), white grouper (Epinephelus aeneus), banded grouper (Epinephelus amblycephalus), areolate grouper (Epinephelus areolatus), duskytail grouper (Epinephelus bleekeri), pale margin grouper (Epinephelus bontoides), brown spotted grouper (Epinephelus chlorostigma), orange-spotted grouper (Epinephelus coiodes), blacktip grouper (Epinephelus fasciatus), brown-marbled grouper (Epinephelus fuscoguttatus), starry grouper (Epinephelus labriformis), giant grouper (Epinephelus lanceolatus), highfin grouper (Epinephelus maculatus), Malabar grouper (Epinephelus malabaricus), dusky grouper (Epinephelus marginatus), white-streaked grouper (Epinephelus ongus), camouflage grouper (Epinephelus polyphekadion), longfin grouper (Epinephelus quoyanus), sixbar grouper (Epinephelus sexfasciatus), Nassau grouper (Epinephelus striatus), greasy grouper (Epinephelus tauvina), and potato grouper (Epinephelus tukula); fishes belonging to the genus Cromileptes such as humpback grouper (Cromileptes altivelis); fishes belonging to the genus Plectropomus such as leopard coralgrouper (Plectropomus leopardus); and crossbreds among fishes of the family Serranidae.

Fishes of the family Cichlidae can include, for example, fishes of the genus Oreochromis such as Nile tilapia (Oreochromis niloticus), Mozambique tilapia (Oreochromis mossambicus), and Blue tilapia (Oreochromis aureus).

Fishes of the family Adrianichthyidae can include, for example, fishes of the genus Oryzias such as medaka (Oryzias latipes, Oryzias sakaizumii) and Javanese ricefish (Oryzias javanicus).

Fishes of the family Monacanthidae can include, for example, fishes of the genus Stephanolepis such as thread-sail filefish (Stephanolepis cirrhifer) and fishes of the genus Thamnaconus such as black scraper (Thamnaconus modestus).

Fishes of the family Osmeridae can include, for example: fishes of the subfamily Plecoglossinae such as ayu (Plecoglossus altivelis); fishes of the subfamily Hypomesinae such as Japanese smelt (Hypomesus nipponensis) and Japanese surfsmelt (Hypomesus japonicus); fishes of the subfamily Osmerinae such as Arctic rainbow smelt (Osmerus mordax dentex), Shishamo smelt (Spirinchus lanceolatus), and Japanese icefish (Salangichthys microdon); and the like.

Fishes of the family Scombridae can include, for example: fishes belonging to the genus Scombrini such as chub mackerel (Scomber japonicus), Atlantic mackerel (Scomber scombrus), and blue mackerel (Scomber australasicus); fishes belonging to the genus Thunnini such as Pacific bluefin tuna (Thunnus orientalis), Atlantic bluefin tuna (Thunnus thynnus), southern bluefin tuna (Thunnus maccoyii), bigeye tuna (Thunnus obesus), yellowfin tuna (Thunnus albacares), albacore (Thunnus alalunga), and longtail tuna (Thunnus tonggol); fishes belonging to the genus Euthynnus such as Kawakawa (Euthynnus affinis) and Little tunny (Euthynnus alletteratus); fishes belonging to the genus Katsuwonus such as skipjack tuna (Katsuwonus pelamis); fishes belonging to the genus Scomberomorini; fishes belonging to the genus Auxis; fishes belonging to the genus Sardini; fishes belonging to the genus Gymnosarda; and the like.

Fishes of the family Pleuronectidae can include, for example, littlemouth flounder (Pseudopleuronectes herzensteini), marbled flounder (Pleuronectes yokohamae), stone flounder (Kareius bicoloratus), Pacific halibut (Hippoglossus stenolepis), barfin flounder (Verasper moseri), and the like.

Fishes of the family Carangidae can include, for example: fishes belonging to the genus Seriola such as greater amberjack (Seriola dumerili), yellowtail amberjack (Seriola lalandi), almaco jack (Seriola rivoliana), and Japanese amberjack (Seriola quinqueradiata); fishes belonging to the genus Pseudocaranx such as Japanese jack mackerel (Trachurus japonicus) and hard-tail jack (Pseudocaranx dentex); fishes belonging to the genus Trachinotus such as snubnose pompano (Trachinotus blochii).

Fishes of the family Lateolabrax can include, for example, blackfin seabass (Lateolabrax latus), spotted seabass (Lateolabrax maculatus), and the like. Fishes of the family Moronidae includes, for example, European seabass (Dicentrarchus labrax) and the like.

Fishes of the family Latidae can include, for example, fishes of the genus Lates such as barramundi perch (Lates calcarifer) and Nile perch (Lates niloticus). Fishes of the family Rachycentridae can include, for example, cobia (Rachycentron canadum) and the like.

Fishes of the family Cynoglossidae can include, for example, red tonguesole (Cynoglossus joyneri), tongue sole (Cynoglossus semilaevis), and the like.

Fishes of the family Anguillidae can include, for example, Japanese eel (Anguilla japonica), European eel (Anguilla anguilla), and the like.

Fishes of the family Congridae can include, for example, whitespotted conger (Conger myriaster), Beach conger (Conger japonicus), and the like.

Preferably, the fishes are of the families Cichlidae and Tetraodontidae, and are a tilapia and a grass puffer.

In the present description, the fishes may be true breds or crossbreds. The crossbreds can include, for example, hybrids derived from intergeneric crossing.

The “fish” is preferably farmed fish. In addition, the “farmed fish” can include, for example, fish that are bred for purposes of food, reproduction, decoration, and the like.

An example of a sterilization gene is a dead end (dnd) gene.

Gene IDs of sterilization genes of major fishes registered in NCBI are listed below:
Taxonomy ID:Organism name:Gene ID
7906:Acipenser ruthenus:131699448
7906:Acipenser ruthenus:117412941
7913:Polyodon spathula:121323773
7913:Polyodon spathula:121297601
7918:Lepisosteus oculatus:102698012
7936:Anguilla anguilla:118224086
7950:Clupea harengus:105903488
7955:Danio rerio:373074
7957:Carassius auratus:113057682
7957:Carassius auratus:113113732
7959:Ctenopharyngodon idella:127494684
7962:Cyprinus carpio:109089193
7962:Cyprinus carpio:109108346
7994:Astyanax mexicanus:103031423
7998:Ictalurus punctatus:108274524
8005:Electrophorus electricus:1182426708005:Electrophorus electricus:1182426698005:Electrophorus electricus:1135817468010:Esox lucius:105029177
8017:Oncorhynchus gorbuscha:123993384
8018:Oncorhynchus keta:118377783
8019:Oncorhynchus kisutch:109864762
8022:Oncorhynchus mykiss:100136693
8023:Oncorhynchus nerka:115130677
8030:Salmo salar:101448053
8030:Salmo salar:106611692
8032:Salmo trutta:115172323
8036:Salvelinus alpinus:111965708
8038:Salvelinus fontinalis:129858071
8040:Salvelinus namaycush:120046990
8049:Gadus morhua:115551867
8078:Fundulus heteroclitus:105920226
8081:Poecilia reticulata:103471153
8083:Xiphophorus maculatus:102223082
8084:Xiphophorus hellerii:116714911
8090:Oryzias latipes:100302723
8103:Cyclopterus lumpus:117737633
8128:Oreochromis niloticus:100712141
8153:Haplochromis burtoni:102308484
8154:Astatotilapia calliptera:1130301828167:Perca flavescens:114562870
8168:Perca fluviatilis:120566341
8175:Sparus aurata:115568425
8177:Acanthopagrus latus:119007972
8187:Lates calcarifer:108895524
8208:Notothenia coriiceps:104953557
8218:Gymnodraco acuticeps:117550442
8236:Thunnus albacares:122990905
8240:Thunnus maccoyii:121902261
8245:Xiphias gladius:120794382
8245:Xiphias gladius:120784879
8255:Paralichthys olivaceus:109627263
8255:Paralichthys olivaceus:109629018
8262:Pleuronectes platessa:128436393
8262:Pleuronectes platessa:128446010
8267:Hippoglossus hippoglossus:1177690828267:Hippoglossus hippoglossus:11776969813013:Clarias gariepinus:128512679
13489:Dicentrarchus labrax:127375252
13676:Scomber japonicus:128363493
27687:Erpetoichthys calabaricus:11466000027706:Micropterus salmoides:119884054
27706:Micropterus salmoides:119889311
28743:Cyprinodon variegatus:107097352
28829:Solea senegalensis:122760098
29144:Chanos chanos:115804993
30732:Oryzias melastigma:112153172
31033:Takifugu rubripes:101063254
32473:Xiphophorus couchianus:114152529
32507:Neolamprologus brichardi:10277558333528:Gambusia affinis:122836594
34773:Alosa sapidissima:121694293
34816:Morone saxatilis:118333920
37003:Kryptolebias marmoratus:10824965740690:Trematomus bernacchii:117470848
41447:Seriola dumerili:111224560
41447:Seriola dumerili:111230405
42514:Pygocentrus nattereri:108442481
42526:Colossoma macropomum:118811266
42526:Colossoma macropomum:118801938
42636:Brienomyrus brachyistius:12575088143689:Simochromis diagramma:120720608
43700:Monopterus albus:109966434
47969:Oreochromis aureus:116317994
48193:Mugil cephalus:125008417
48698:Poecilia formosa:103154733
48699:Poecilia latipinna:106940460
48701:Poecilia mexicana:106910880
52239:Pseudochaenichthys georgianus:11745418952670:Austrofundulus limnaeus:10652439152904:Scophthalmus maximus:118319583
52904:Scophthalmus maximus:118312266
54343:Etheostoma spectabile:116697245
55291:Polypterus senegalus:120543184
56716:Cottoperca gobio:115014268
56723:Labrus bergylta:109994465
59861:Coregonus clupeaformis:121585904
59861:Coregonus clupeaformis:121542650
63155:Archocentrus centrarchus:11578722563155:Archocentrus centrarchus:11578721864144:Anabas testudineus:113150433
64144:Anabas testudineus:113160572
66913:Ictalurus furcatus:128618702
69293:Gasterosteus aculeatus:120816881
70543:Myxocyprinus asiaticus:127417548
70543:Myxocyprinus asiaticus:127412725
72105:Sebastes umbrosus:119493801
74940:Oncorhynchus tshawytscha:11222092675038:Scatophagus argus:124067992
75329:Misgurnus anguillicaudatus:12942252075352:Megalobrama amblycephala:12525910275366:Sinocyclocheilus grahami:10758708975366:Sinocyclocheilus grahami:10755929277115:Cyprinodon tularosa:119773491
80966:Acanthochromis polyacanthus:11097021780966:Acanthochromis polyacanthus:11094679780972:Amphiprion ocellaris:111570639
84645:Labeo rohita:127175905
90069:Solea solea:131472944
90988:Pimephales promelas:120460971
101364:Carassius gibelio:127971074
101364:Carassius gibelio:128027787
105023:Nothobranchius furzeri:107373510106582:Maylandia zebra:101485786
109280:Hippocampus comes:109525804
109293:Hippocampus zosterae:127599348
109905:Chelmon rostratus:121611674
113540:Scleropages formosus:108936068
113540:Scleropages formosus:108923024
118141:Megalops cyprinoides:118790659
119488:Siniperca chuatsi:122880547
134920:Pungitius pungitius:119210979
137520:Hypomesus transpacificus:124470547144197:Stegastes partitus:103363223
147949:Micropterus dolomieu:123958144
150288:Boleophthalmus pectinirostris: 110159042154827:Xyrauchen texanus:127663360
154827:Xyrauchen texanus:127659470
158456:Betta splendens:114864992
160734:Plectropomus leopardus:121948100161448:Corythoichthys intestinalis:130926506161448:Corythoichthys intestinalis:130927109161450:Doryrhamphus excisus:131110374
161453:Dunckerocampus dactyliophorus:129189966161584:Syngnathus acus:119121031
161590:Syngnathus scovelli:125967294
173247:Echeneis naucrates:115037193
173247:Echeneis naucrates:115049621
175797:Silurus meridionalis:124392454
181472:Salarias fasciatus:115401214
188132:Poeciliopsis prolifica:129353865195615:Hippoglossus stenolepis:118118622195615:Hippoglossus stenolepis:118112154205130:Mastacembelus armatus:113144127
208333:Girardinichthys multiradiatus:124860564210632:Parambassis ranga:114442990
215358:Larimichthys crocea:104935689
229290:Anoplopoma fimbria:129090164
241271:Cheilinus undulatus:121515885
244447:Cynoglossus semilaevis:103390515270530:Synchiropus splendidus:128767449278164:Alosa alosa:125286341
283035:Sander lucioperca:116064383
293821:Epinephelus fuscoguttatus:125894322299321:Denticeps clupeoides:114768310
300413:Epinephelus moara:126389345
303518:Pundamilia nyererei:102198131
307959:Sinocyclocheilus rhinocerous:107758536307959:Sinocyclocheilus rhinocerous:107731723310571:Epinephelus lanceolatus:117260757310915:Pangasianodon hypophthalmus:113529581337641:Hemibagrus wyckioides:131365133
369639:Onychostoma macrolepis:131553405375764:Sphaeramia orbicularis:115416804375764:Sphaeramia orbicularis:115427540409849:Periophthalmus magnuspinnatus:117377737417921:Etheostoma cragini:117952205
433405:Anarrhichthys ocellatus:116400932433684:Takifugu flavidus:130531798
441366:Gouania willdenowi:114471152
451745:Nematolebias whitei:119408307
586833:Myripristis murdjan:115371519
586833:Myripristis murdjan:115366093
941984:Toxotes jaculatrix:121193628
941984:Toxotes jaculatrix:121193618
941984:Toxotes jaculatrix:121188811
992332:Triplophysa rosa:130564032
1042646:Gadus chalcogrammus:130390898
1142201:Danio aesculapii:130240883
1203425:Notolabrus celidotus:117817455
1234273:Tachysurus fulvidraco:1136548571250792:Melanotaenia boesemani:1216431261582913:Triplophysa dalaica:130438038
1606681:Puntigrus tetrazona:122357788
1608454:Sinocyclocheilus anshuiensis:1076809071608454:Sinocyclocheilus anshuiensis:1076601971676925:Paramormyrops kingsleyae:1118529871841481: Seriola lalandi dorsalis:1116600411841481:Seriola lalandi dorsalis:1116635992059687:Pseudoliparis swirei:130209981
2546036:Lampris incognitus:130110548
2546036:Lampris incognitus:130110540
2871759:Seriola aureovittata:130163207
2871759:Seriola aureovittata:130173671
3034132:Rhinichthys klamathensis goyatoka:130084764.

The fluorescent protein transfected into the sterilization gene allele is not limited. Examples are: green fluorescent proteins such as TurboGFP, AcGFP, TagGFP, Azami-Green, ZsGreen, EmGFP, EGFP, GFP2, HyPerGFP, PS-CFP, Dendra2, Kaede, EosFP, and KikumeGR; blue fluorescent proteins such as Sirius and EBFP; cyan fluorescent proteins such as ECFP, mTurquoise, TagCFP, AmCyan, mTFP1, MidoriishiCyan, CFP, and PS-CFP; yellow fluorescent proteins such as TagYFP, EYFP, Venus, YFP, PhiYFP, PhiYFP-m, TurboYFP, ZsYellow, and mBanana; orange fluorescent proteins such as KusabiraOrange and mOrange; red fluorescent proteins such as TurboRFP, DsRed-Express, DsRed2, TagRFP, DsRed-Monomer, AsRed2, mStrawberry, Dendra2, Kaede, EosFP, and KikumeGR; and farred fluorescent proteins such as TurboFP602, mRFP1, JRed, KillerRed, mCherry, HcRed, KeimaRed, mRasberry, and mPlum.

In a case of the homo-type, the polynucleotide sequence encoding the fluorescent protein with the same fluorescent wavelength band may be transfected into both of the sterilization gene alleles, or the polynucleotide sequence encoding the fluorescent protein with a different fluorescent wavelength band may be transfected into each of the sterilization gene alleles.

The fluorescent proteins having the same fluorescence wavelength band are intended to mean that a difference in peak fluorescence wavelength emitted by the two fluorescent proteins is within, for example, 30 nm, preferably 20 nm, when the two fluorescent proteins are irradiated with excitation light having an appropriate wavelength.

The fluorescent proteins having the different fluorescence wavelength bands are intended to mean that the difference in peak fluorescence wavelength emitted by the two fluorescent proteins is, for example, 20 nm or greater, preferably 30 nm or greater, more preferably 50 nm or greater when the two fluorescent proteins are irradiated with the excitation light having the appropriate wavelength.

The excitation light having the appropriate wavelength is intended to mean the excitation light recommended for the respective fluorescent protein.

The term “polynucleotide sequence encoding the fluorescent protein to be expressible” is intended to mean that the fluorescent protein can be expressed from the polynucleotide sequence encoding the fluorescent protein under control of a promoter.

The polynucleotide sequence encoding the fluorescent protein is linked directly or indirectly to the 3′-downstream region of a promoter sequence for expressing the fluorescent protein. The term “indirectly” is intended to mean that a spacer sequence of 1 nucleotide (nt) to 20 nt may be included.

The promoter is not limited as long as the promoter exhibits promoter activity in the fish individual. The promoter may control tissue-specific expression, or may control systemic expression. The promoter may control tissue-specific expression, or may control systemic expression. The tissue-specific expression of the fluorescent protein preferably occurs at a site, such as an eye, a scale, skin, or a fin, that can be observed from a body surface. Examples of the promoter that controls the tissue-specific expression are a gamma crystallin, an alpha crystallin, a beta crystallin, a delta crystallin, rhodopsin, RPE 65, IRBP, and an arrestin gene, which control the expression in the eyes. Examples of the promoter that controls the tissue-specific expression are sp7, keratin, and a KRT5 gene. An example of the promoter that controls the systemic expression is actin.

The hetero type and the homo type as phenotypes can be confirmed by confirmation of the expression of the fluorescent protein. The fluorescent protein can be expressed by irradiating an embryonic or adult body surface of the individual fish with the appropriate excitation light and observing the fluorescence. The fluorescence may be observed by human eyes or by a fluorescence microscope or a microscope. Alternatively, a computer may be used for observation. In the case of the hetero type, a human or the computer can determine presence or absence of the fluorescent protein, and if the fluorescent protein is expressed, the individual can be determined as the hetero type. In the case of the homo type, the individual can be determined as the homo type when the following condition is satisfied. Both of the sterilization gene alleles have the polynucleotide sequence encoding the fluorescent protein having the same fluorescent wavelength band, and intensity of the fluorescence is greater than that of the hetero type. In addition, in the case where the sterilization gene alleles have the polynucleotide sequences encoding the fluorescent proteins having the different fluorescent wavelength bands from each other, and the fluorescent proteins are expressed, the individual can be determined as the homo type. At the time, the embryonic body of the individual fish or the adult fish can be maintained in a viable state.

### 2. Method for Producing Fish Individual Deficient in at Least Part of Function of Sterilization Protein

An embodiment relates to a method for producing the fish individual deficient in at least a part of the function of the sterilization protein (hereinafter also simply referred to as a “production method”). The production method includes transfecting the polynucleotide sequence encoding the fluorescent protein (hereinafter also simply referred to as a “fluorescent protein sequence”) to be expressible into at least one of the sterilization gene alleles encoding the sterilization protein.

In this section, the description of the terms used in Section 1. above is hereby incorporated by reference.

For example, as described in Section 1. above, as a donor cassette polynucleotide linked directly or indirectly to the 3′-downstream region of the promoter sequence, the fluorescent protein sequence can be transfected into the sterilization gene allele. The fluorescent protein sequence is preferably a cDNA sequence of the fluorescent protein. The donor cassette polynucleotide preferably has a polyA signal in addition to the promoter sequence and the fluorescent protein sequence.

The donor cassette polynucleotide can be transfected into the sterilization gene allele by a known method.

Examples of the transfection method are a genome editing method or a knock-in method using a gene site-specific recombination method.

The genome editing method can include a method for transfecting a protein and a nucleic acid having a genome editing technique or a vector encoding these into a fertilized egg. An example of the protein can include a clustered regularly interspaced short palindromic repeats (CRISPR) enzyme. More specifically, examples of the CRISPR enzyme can include Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4. Examples of the nucleic acid can include crRNA and tracrRNA, or a single-stranded nucleic acid linking these via a linker. In this case, for example, the nucleic acid is designed such that a base sequence to anneal to a target sequence in the crRNA contains a base sequence encoding the sterilization gene and a complementary base sequence. A single type of the nucleic acid may be used alone, or two or more types of the nucleic acids may be used in combination. The genome editing system using the CRISPR enzyme can include a clustered regularly interspaced short palindromic repeats/CRISPR associated protein 9 (CRISPR/Cas9) system. Another genome editing system can include at least one method selected from the CompoZr zinc finger nuclease (ZFN) system and the TAL effector nuclease (TALEN) system. Preferably, the genome editing system is the CRISPR/Cas9 system that transfects Cas9 as mRNA or a protein and transfects gRNA in a state of being sgRNA or crRNA and tracrRNA. In a vector-based CRISPR/Cas9 system, the nucleic acid encoding CRISPR and the nucleic acid encoding Cas9 may be on different vectors or on one vector. The promoter for a CRISPR function is not particularly limited, but is preferably a U6 promoter. The promoter for a Cas9 function is not particularly limited, but is preferably a promoter expressed in a mammalian cell, such as a Cytomegalovirus promoter. As the CRISPR/Cas9 system, preferably a commercially available vector such as a pX330-U6-Chimeric_BB-CBh-hSpCas9 vector can be used.

A sequence that is incorporated into a CRISPR sequence and targets the sterilization gene (hereafter referred to as the “target sequence”) is not limited as long as such a sequence can be incorporated into a guide RNA (also referred to as gRNA, sgRNA, or crRNA) and transcribed by the CRISPR/Cas9 system, or can be transfected as the guide RNA containing the complementary sequence in the target sequence into a cell and recombine the sterilization gene. In general, it is said to be able to select, as the target sequence, a sequence of about 20 bases in a 5′-upstream region of a base sequence “NGG” (a PAM sequence: N is any of nucleotide A, G, T, C) present in the sterilization gene. The target sequence can be designed by using any of known design tools published by an Optimized CRISPR design tool (a website of Zhang Lab at Massachusetts Institute of Technology (http://crispr.mit.edu/)), E-CRISP (http://www.e-crisp.org/E-CRISP/ (German Cancer Research Center)), ZiFiT Targeter (http://zifit.partners.org/ZiFit/ (Zing Finder Consortium)), Cas9 design (http://cas9.cbi.pku.edu.cn (Peking University)), CRISPRdirect (http://crispr.dbcls.jp (the University of Tokyo)), CRISPR-P (http://cbi.hzau.edu.cn/crispr/ (Huazhong Agricultural University)), CRISPR RGEN Tools (http://www.rgenome.net/ (Seoul National University)), and the like.

In addition, preferably, when single nucleotide polymorphisms (SNPs) exist in the PAM sequence, such a sequence is preferably avoided. In regard to the target sequence, when the SNPs of an individual are known, the sequence is preferably optimized for each of the SNPs.

In a 5′-terminal region of the target sequence, the sequence may be one, two, three, or four bases shorter, preferably, one, two, or three bases shorter.

The CRISPR/Cas9 system may also be used to transfect the gene into the cell as the vector, or gRNA, crRNA, trans-activating crRNA (tracrRNA), and RNA encoding Cas9 synthesized by artificial synthesis or by transcription in vitro may be combined and transfected into the cell. Alternatively, a combination of a Cas9 protein and the guide RNA may be transfected into the cell.

Furthermore, in the genome editing system, a donor oligo DNA such as a single-stranded oligos (ssODNs) may be co-transfected into the cell. The ssODNs can be designed according to a known method.

The genome editing system can perform microinjection into cytoplasm of a fertilized egg, preferably, a fertilized egg at a one-cell stage. For example, when being transfected, the Cas9 protein can be injected in a range of 5 pg to 100 pg, preferably 10 pg to 80 pg, more preferably 10 pg to 50 pg per fertilized egg. At this time, the guide RNA can be injected in a range of 0.1 pg to 50 pg, preferably 0.5 pg to 20 pg, more preferably 1 pg to 5 pg. Example 1 described below can be referred for a method for introducing a mutation using the genome editing technique, for example.

For example, as the gene site-specific recombination method can be a method using at least one selected from a Cre/loxP system, a Flp/FRT system, a Dre/rox system, and a PhiC31 integrase system, and a variant of the gene site-specific recombination method.

### 3. Method for Identifying Genotype

An embodiment relates to a method for identifying a genotype (hereinafter also simply referred to as an “identification method”). The method includes: mating male and female fish individuals, each of which has the polynucleotide sequence encoding the fluorescent protein to be expressible only in one of the sterilization gene alleles of the fish individual described in Section 1. above; and observing the expression of the fluorescent protein in the embryonic body or adult fish produced by mating.

In the identification method, sexes to be mated may be the same species or different species that can interbreed.

When the sexes of the fish individuals in the hetero type described in Section 1. above are mated, any of three types of the wild type, the hetero type, and the homo type can appear as the genotype of the next generation according to Mendel's laws of inheritance. These are identified by the expression of the fluorescent protein.

The observation of the expression of the fluorescent protein and the method for determining whether the individual is of the hetero type or the homo type are as described in Section 1. above. In a case of the wild type, the expression of the fluorescent protein is not observed.

### 4. Method for Transplanting Germ Cell

An embodiment relates to a method for transplanting a germ cell, the method including transplanting a germ cell of a different fish species from the fish individual into a fish individual of the homo type described in Section 1. above.

Here, the different fish species from the fish individual of the homo type described in Section 1. above is not limited as long as such a fish species is adaptable to a fish body of the fish individual of the homo type.

The transplantation method is known. For example, the transplantation method is a method for producing a gamete of the fish using a surrogate parent fish technique. Donor undifferentiated germ cells that are used in the surrogate parent fish technique include a primordial germ cell obtained from a gonad prior to sexual differentiation, a spermatogonium obtained from testes, an oogonium obtained from ovaries, and the like.

In order to obtain the undifferentiated germ cell from the donor, a normal method can be used to collect the undifferentiated germ cell from the donor's tissue according to a differentiation stage of the desired undifferentiated germ cell. For example, the undifferentiated germ cell can be obtained by harvesting the gonad prior to the sexual differentiation or the tissue after the sexual differentiation, such as the testes or the ovaries, from the donor and dispersing the gonad or the tissue into individual cells by physical detachment or treatment using a proteolytic enzyme. The individual dispersed cells can be isolated, for example, by using an antibody as a marker or using a cell sorter.

The undifferentiated germ cell can be obtained from a frozen or live individual. In order to increase a success rate of the surrogate parent fish technique, it is preferable to obtain the undifferentiated germ cell from the live individual.

When the donor cell is transfected into a recipient, it is preferred to introduce the cell into the embryo or the individual at a hatchling stage before the recipient's immune system is fully engaged. The transfection can be performed by using a manipulator such as a micromanipulator, an electrocautery scalpel, or a laser scalpel. The transfection may be performed in any tissue or site of the recipient, and examples of the tissue or the site as the transfection target include an epidermis and the abdominal cavity. The number of the cells to be transfected into the embryo or the individual at the hatchling stage is not particularly limited, and 1 to 100,000 cells can be transfected, for example.

### EXAMPLES

Hereinafter, a detailed description will be made on the present invention by using examples. However, the present invention is not to be construed as limited to the examples. The sequences corresponding to the sequence numbers used in the examples will be provided in the sequence list below.

### I. Preparation of Cassette Polynucleotide for Expression of Fluorescent Protein

First and second cassette polynucleotides capable of expressing the fluorescent proteins under the control of a crystallin promoter were prepared. The first cassette polynucleotide has an EGFP cDNA sequence (sequence number 2) downstream of a mouse gamma-crystallin promoter (sequence number 1) and a Simian virus 40 (SV40) polyA signal (sequence number 3) further downstream. Hereinafter, this cassette polynucleotide will be referred to as Crystallin promoter-EGFP-SV40 polyA, and the polynucleotide sequence has the sequence number 4. The second cassette polynucleotide has an mCherry cDNA sequence (sequence number 5) downstream of the mouse gamma-crystallin promoter (the sequence number 1) and the SV40 polyA signal (the sequence number 3) further downstream. Hereafter, this donor cassette polynucleotide will be referred to as Crystallin promoter-mCherry-SV40 polyA, and a polynucleotide sequence has the sequence number 6.

### II. Example Using Tilapia as Test Species

### 1. Preparation of Donor Construct

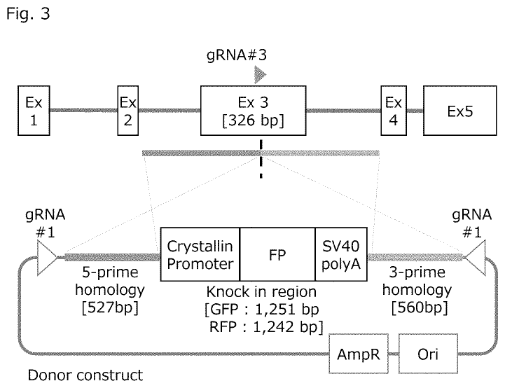

To label a germ-cell deficient individual by the genome editing method using a tilapia as a test species, a donor construct was prepared to transfect the cassette polynucleotide described in Section I. above into the tilapia. A sequence map of the donor construct is shown in FIG. 3.
(1) 5-prime Onil dnd homology having the sequence indicated by the sequence number 7 and 3-prime Onil dnd homology having the sequence indicated by the sequence number 8 were isolated from tilapia dnd (sequence number 9).
(2) A gRNA #1 guide RNA sequence indicated by the sequence number 10 for vector cleavage, the 5-prime Onil dnd homology sequence indicated by the sequence number 7, the Crystallin promoter-EGFP-SV40 polyA sequence indicated by the sequence number 4, the 3-prime Onil dnd homology sequence indicated by the sequence number 8, gRNA #1 indicated by the sequence number 10, an Ori sequence indicated by the sequence number 11 as the origin of plasmid replication in E. coli, and an Onil Crystallin promoter-EGFP-SV40 polyA vector (the nucleotide sequence being indicated by the sequence number 13) having an AmpR sequence of an ampicillin resistance gene indicated by the sequence number 12 were prepared. In addition, an Onil Crystallin promoter-mCherry-SV40 polyA vector, into which Crystallin promoter-mCherry-SV40 polyA indicated by the sequence number 6 (the nucleotide sequence being indicated by the sequence number 14) was transfected instead of Crystallin promoter-EGFP-SV40 polyA indicated by the sequence number 4, was prepared.
   2. Transfection of Cassette Polynucleotide into Dnd Gene Region and Confirmation Test
   (1) Transfection of Cassette Polynucleotide into Dnd Gene Region

The Onil Crystallin promoter-EGFP-SV40 polyA vector indicated by the sequence number 13 and the Onil Crystallin promoter-mCherry-SV40 polyA vector indicated by the sequence number 14 were used to transfect the cassette polynucleotide into the tilapia genome by the knock-in method of the genome editing method using the CRISPR-Cas9 system.

Cas9 protein 250 ng/μl; gRNA #3 (the sequence number16) targeting exon 3 (the sequence number 15) of the tilapia dnd gene 50 ng/μl; the Onil Crystallin promoter-EGFP-SV40 polyA vector or the Onil Crystallin promoter-mCherry-SV40 polyA vector 2.5 ng/μl; gRNA #1 (the sequence number 10) 50 ng/μl that cleaves the construct were microinjected into the fertilized tilapia eggs.

### (2) Confirmation of Expression of Fluorescent Protein

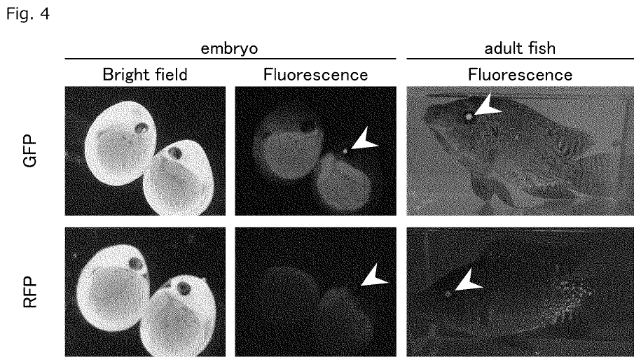

The fertilized tilapia eggs that had been subjected to the microinjection were incubated at 28° C., and the expression of GFP or mCherry was observed in 4 to 6-day embryos using a stereo microscope. The expression of the fluorescent protein in the eyes of the embryonic body and the adult fish produced from the fertilized tilapia eggs, which had been subjected to the microinjection, was confirmed (FIG. 4).

In order to confirm that the cassette polynucleotide was correctly transfected into the dnd region, genome DNA was extracted from the individuals, in which the fluorescence was observed in the above observation, and the genome sequence was checked by the PCR method and nucleotide sequence sequencing.

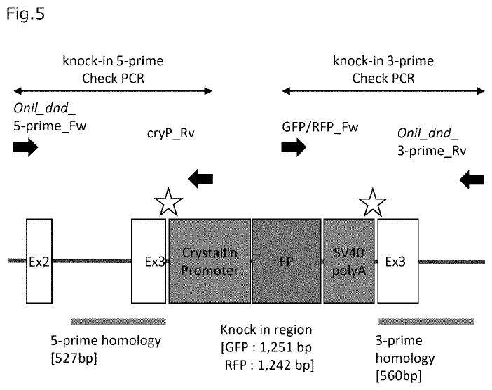

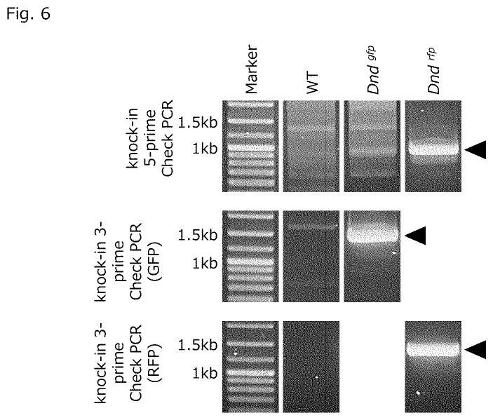

As shown in FIG. 5, the PCR primer was designed outside a region of knock-in homology using CRISPR-Cas9 and between the cassette polynucleotides. Star symbols in FIG. 5 each indicate a confirmation site. In order to confirm the 5′-side (the upstream side), PCR was performed using Onil_dnd_5-prime_Fw (the sequence number 17) and cryP_Rv (the sequence number 18). In addition, in order to confirm the 3′-side (the downstream side), PCR was performed using GFP_Fw (the sequence number 19) and Onil_dnd_3-prime_Rv (the sequence number 20) for a dnd^{gfp} sample, and RFP_Fw (the sequence number 21) and Onil_dnd_3-prime_Rv (the sequence number 20) for a dnd^{rfp} sample. Each PCR product was electrophoresed on a 1.5% agarose gel to check presence or absence and a size of the band and thereby confirmed that the desired amplified product was obtained (FIG. 6).

The above PCR products were subcloned and subjected to the Sanger sequencing analysis using an Onil_dnd_5-prime_Fw primer and an Onil_dnd_3-prime_Rv primer, respectively.

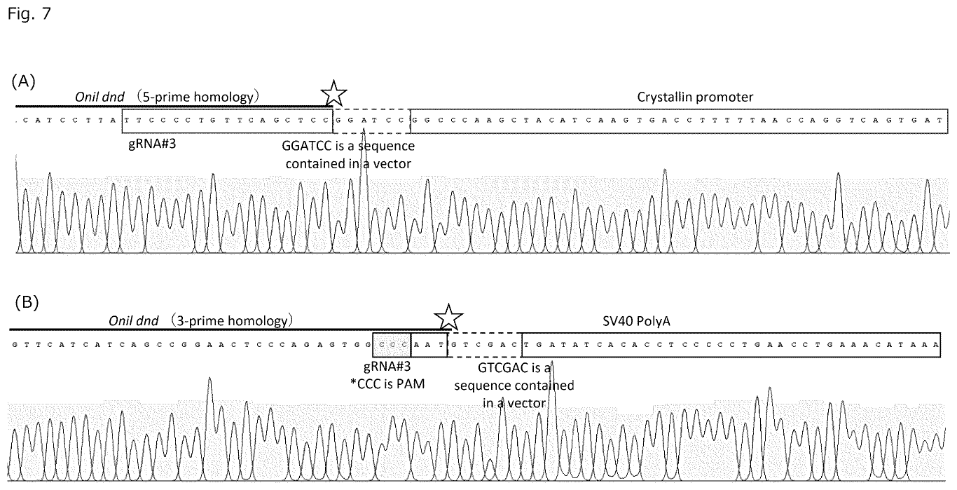

FIG. 7 shows sequences at the cassette polynucleotide transfection sites in the dnd^{gfp} individual. FIG. 7A shows the 5′-side sequence analyzed by using the Onil_dnd_5-prime_Fw primer. FIG. 7B shows the 3′-side sequence analyzed by using the Onil_dnd_3-prime_Rv primer.

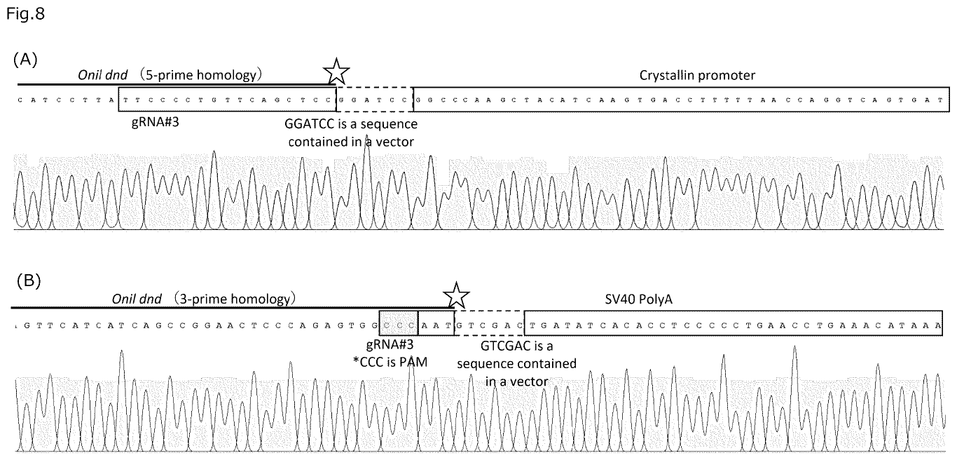

FIG. 8 shows sequences at the cassette polynucleotide transfection sites in the dnd^{rfp} individual. FIG. 8A shows the 5′-side sequence analyzed by using the Onil_dnd_5-prime_Fw primer. FIG. 8B shows the 3′-side sequence analyzed by using the Onil_dnd_3-prime_Rv primer.

It was shown that the cassette polynucleotide was correctly inserted in the target sites by the PCR method and sequencing.

### III. Example Using Grass Puffer as Test Species

### 1. Preparation of Donor Construct

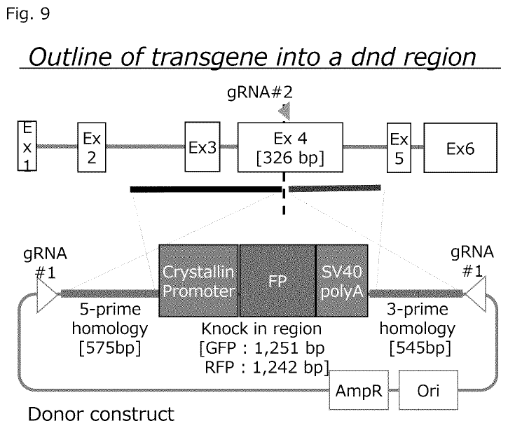

To label a germ-cell deficient individual by the genome editing method using a grass puffer as a test species, a donor construct was prepared to transfect the cassette polynucleotide described in Section I. above into the grass puffer. A sequence map of the donor construct is shown in FIG. 9.
(1) 5-prime Talb dnd homology (the sequence number 22) and 3-prime Talb dnd homology (the sequence number 23) were isolated from grass puffer dnd (the sequence number 24).
(2) The gRNA #1 guide RNA sequence indicated by the sequence number 10 for vector cleavage, the 5-prime Talb dnd homology sequence indicated by the sequence number 22, the Crystallin promoter-EGFP-SV40 polyA sequence indicated by the sequence number 4, the 3-prime Talb dnd homology sequence indicated by the sequence number 23, gRNA #1 indicated by the sequence number 10, the Ori sequence indicated by the sequence number 11, and a Talb Crystallin promoter-EGFP-SV40 polyA vector (the nucleotide sequence being indicated by the sequence number 25) having the AmpR sequence indicated by the sequence number 12 were prepared. In addition, a Talb Crystallin promoter-mCherry-SV40 polyA vector (the nucleotide sequence being indicated by the sequence number 26), into which Crystallin promoter-mCherry-SV40 polyA indicated by the sequence number 6 was transfected instead of Crystallin promoter-EGFP-SV40 polyA indicated by the sequence number 4, was prepared.
   2. Transfection of Cassette Polynucleotide into Dnd Gene Region and Confirmation Test
   (1) Transfection of Cassette Polynucleotide into Dnd Gene Region

The Talb Crystallin promoter-EGFP-SV40 polyA vector indicated by the sequence number 25 and the Talb Crystallin promoter-mCherry-SV40 polyA vector indicated by the sequence number 26 were used to transfect the cassette polynucleotide into the grass puffer genome by the knock-in method of the genome editing method using the CRISPR-Cas9 system.

The Cas9 protein 500 ng/μl; gRNA #3 (the sequence number28) targeting exon 4 (the sequence number 27) of the grass puffer dnd gene 50 ng/μl; the Talb Crystallin promoter-EGFP-SV40 polyA vector or the Talb Crystallin promoter-mCherry-SV40 polyA vector 2.5 ng/μl; gRNA #1 (the sequence number 10) 50 ng/μl that cleaves the construct were microinjected into the fertilized grass puffer eggs.

### (2) Confirmation of Expression of Fluorescent Protein

The fertilized grass puffer eggs that had been subjected to the microinjection were incubated at 20° C., and the expression of GFP or RFP was observed in 3 to 6-day embryos using the stereo microscope. The expression of the fluorescent protein in the eyes of the embryonic body and the adult fish produced from the fertilized grass puffer eggs, which had been subjected to the microinjection, was confirmed.

In order to confirm that the cassette polynucleotide was correctly transfected into the dnd region, genome DNA was extracted from the individuals, in which the fluorescence was observed in the above observation, and the genome sequence was checked by the PCR method and nucleotide sequence sequencing.

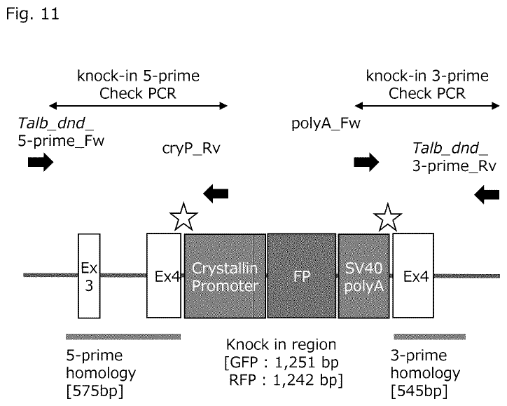

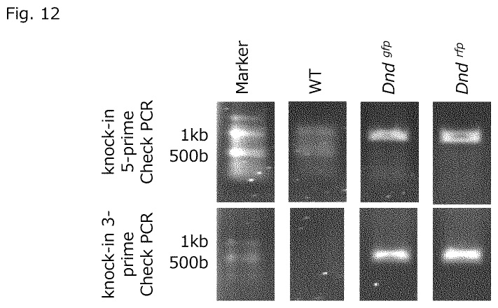

As shown in FIG. 11, the PCR primer was designed outside a region of knock-in homology using CRISPR-Cas9 and between the cassette polynucleotides. Star symbols in FIG. 11 each indicate a confirmation site. In order to confirm the 5′-side (the upstream side), PCR was performed using Talb_dnd_5-prime_Fw (the sequence number 29) and cryP_Rv (the sequence number 18). In addition, in order to confirm the 3′-side (the downstream side), PCR was performed using polyA-Fw (the sequence number 30) and Talb_dnd_3-prime_Rv (the sequence number 31). Each PCR product was electrophoresed on a 1.5% agarose gel to check presence or absence and a size of the band and thereby confirmed that the desired amplified product was obtained (FIG. 12).

The above PCR products were subcloned and subjected to the Sanger sequencing analysis using a Talb_dnd_5-prime_Fw primer and a Talb_dnd_3-prime_Rv primer, respectively.

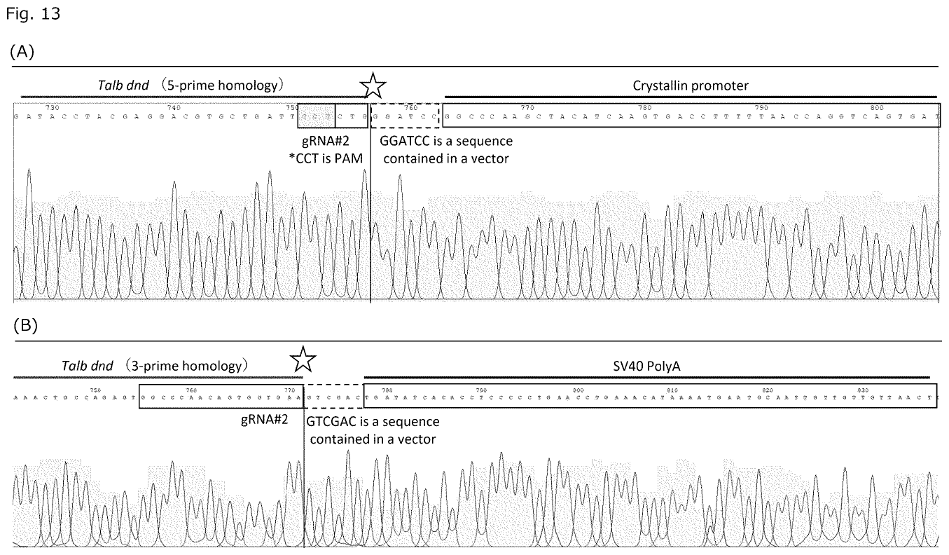

FIG. 13 shows sequences at the cassette polynucleotide transfection sites in the dnd^{gfp} individual. FIG. 13A shows the 5′-side sequence analyzed by using the Talb_dnd_5-prime_Fw primer. FIG. 13B shows the 3′-side sequence analyzed by using the Talb_dnd_3-prime_Rv primer.

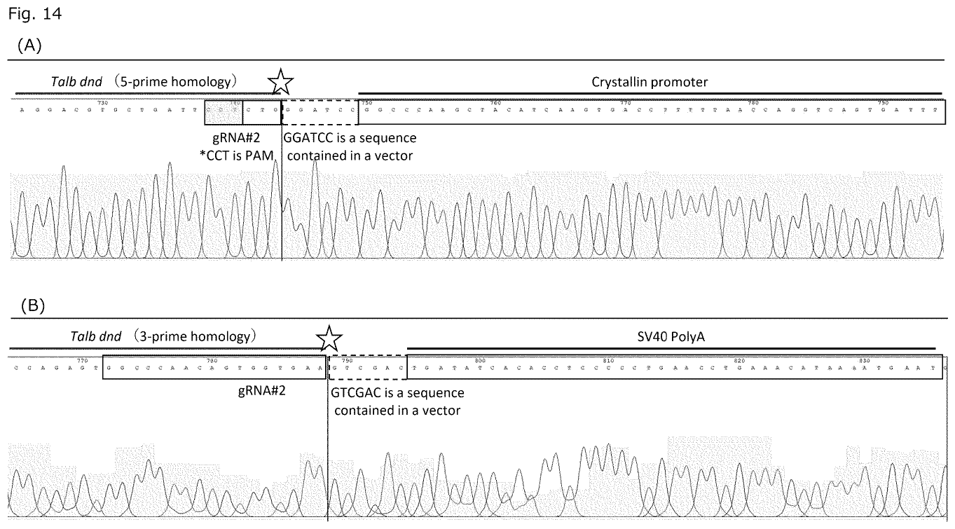

FIG. 14 shows sequences at the cassette polynucleotide transfection sites in the dnd^{rfp} individual. FIG. 14A shows the 5′-side sequence analyzed by using the Talb_dnd_5-prime_Fw primer. FIG. 14B shows the 3′-side sequence analyzed by using the Talb_dnd_3-prime_Rv primer.

It was shown that the cassette polynucleotide was correctly inserted in the target sites by the PCR method and sequencing.

### SEQUENCE LISTING

P24-150WO_PCT_Sterizlization_Protein_Small_20240809_125926_0.xml

1. A fish individual deficient in at least a part of a function of a sterilization protein, the fish individual comprising, in at least one of sterilization gene alleles encoding the sterilization protein which indicates sterility by at least the part of the function thereof being deficient, a polynucleotide sequence encoding a fluorescent protein to be expressible.

2. The fish individual according to claim 1 having the polynucleotide sequence encoding the fluorescent protein in both of the sterilization gene alleles and being deficient in reproductive capacity.

3. The fish individual according to claim 2 having polynucleotide sequences encoding the fluorescent protein and having the same fluorescent wavelength band in both of the sterilization gene alleles.

4. The fish individual according to claim 2 having a polynucleotide sequence encoding the fluorescent protein and having the different fluorescent wavelength band in each of the sterilization gene alleles.

5. The fish individual according to claim 1, wherein
the sterilization gene is a dead end (dnd) gene.

6. The fish individual according to claim 1, wherein
an expression site of the fluorescent protein is an eye.

7. A production method of a fish individual deficient in at least a part of a function of a sterilization protein, which indicates sterility by at least the part of the function thereof being deficient, the method comprising:
transfecting a polynucleotide sequence encoding the fluorescent protein to be expressible in at least one of sterilization gene alleles encoding the sterilization protein.

8. The production method according to claim 7, wherein
the transfection is performed by a genome editing method or a knock-in method using a gene site-specific recombination method.

9. The production method according to claim 8, wherein
the genome editing method is a method using at least one system selected from a clustered regularly interspaced short palindromic repeats/CRISPR associated protein 9 (CRISPR/Cas9) system, CompoZr zinc finger nuclease (ZFN) system, and a TAL effector nuclease (TALEN) system.

10. The production method according to claim 8, wherein
the gene site-specific recombination method is a method using at least one system selected from a Cre/loxP system, a Flp/FRT system, a Dre/rox system, and a PhiC31 integrase system.

11. A polynucleotide comprising:
a polynucleotide sequence encoding a fluorescent protein to be expressible in a part of a sequence of a sterilization gene encoding a sterilization protein, which indicates sterility by at least a part of a function thereof being deficient.

12. A method for transplanting a germ cell, the method comprising:
transplanting a germ cell of a different fish species from the fish individual according to claim 2 into the fish individual.

13. A method for identifying a genotype, the method comprising:
mating male and female fish individuals, each of which has a polynucleotide sequence encoding a fluorescent protein to be expressible only in one of sterilization gene alleles of the fish individual according to claim 1; and
observing expression of the fluorescent protein in an embryonic body or adult fish produced by mating.


SEQUENCE LISTING (31 sequences)

SEQ ID NO: 1 - DNA, 275 bp, Mus musculus
  source 1..275: mol_type = genomic DNA
ggcccaagctacatcaagtgacctttttaaccaggtcagtgatttgtcatccattcgaaagctctggagagtctaagataaaacatacaataacccacacctcccccatagtcattatatagaaaagagaggggaaaaaaagtgccctgtcccactgcgggccccttttgtgctgttcctgccaacacagcagacctcctgctatatagaccctgctcccagccccacacactcaacagcaccatcccatccgacctgccaacaccagccaagct

SEQ ID NO: 2 - DNA, 720 bp, Aequorea victoria
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 3 - DNA, 186 bp, Simian virus 40
  source 1..186: mol_type = genomic DNA
agacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgatatca

SEQ ID NO: 4 - DNA, 1239 bp, synthetic construct
  source 1..1239: mol_type = other DNA
ggcccaagctacatcaagtgacctttttaaccaggtcagtgatttgtcatccattcgaaagctctggagagtctaagataaaacatacaataacccacacctcccccatagtcattatatagaaaagagaggggaaaaaaagtgccctgtcccactgcgggccccttttgtgctgttcctgccaacacagcagacctcctgctatatagaccctgctcccagccccacacactcaacagcaccatcccatccgacctgccaacaccagccaagcttcccatcgattcgaattcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgatatca

SEQ ID NO: 5 - DNA, 711 bp, Discosoma sp.
  source 1..711: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtag

SEQ ID NO: 6 - DNA, 1230 bp, synthetic construct
  source 1..1230: mol_type = other DNA
ggcccaagctacatcaagtgacctttttaaccaggtcagtgatttgtcatccattcgaaagctctggagagtctaagataaaacatacaataacccacacctcccccatagtcattatatagaaaagagaggggaaaaaaagtgccctgtcccactgcgggccccttttgtgctgttcctgccaacacagcagacctcctgctatatagaccctgctcccagccccacacactcaacagcaccatcccatccgacctgccaacaccagccaagcttcccatcgattcgaattcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagtctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgatatca

SEQ ID NO: 7 - DNA, 527 bp, Oreochromis niloticus
  source 1..527: mol_type = other DNA
cttcacagcagtagtgccaatactgcctattctcttcagcattaagaaatatggagttttttggctcagaggtcttcagcatttcaccactgtcaccaagtgccaataatgatatagatttctcaaaggtaaaagttaatcagctgcctttgtttgtaagatgcagctgtgaaaagacaaatctctaaaagctaaatgtgcaaatacttgatggcactgacctaaaactgaacccctgcttgatcttatttggagctgaaacaagaaaaagcccggcttgctcaagcgggtcacatgtcactctattgtaaagacttggacttgctttgtaaagtgcactaaggacttgttgtaaattttgcatcgcatgaacttgaattttaaaattgcataatcagccatcccatttcctctctgtttcatggttagtgtgggaaggtcccacaccgggaccgcgctgtgaagtcttcatcagccagatcccacgggacacgtatgaggacatccttattcccctgttcagctcc

SEQ ID NO: 8 - DNA, 560 bp, Oreochromis niloticus
  source 1..560: mol_type = other DNA
attgggccactctgggagttccggctgatgatgaacttcagtgggcagaaccgcggctttgcgtatgccaaatatggctcagctgctatagctgttgaagccatacgacagctgcacggtcacatggtggagcctggctaccgcatcagtgtacggcggagcacagagaagcgacacctttgtattggaggtctgcctgcttccactagacaagaaggcatactgcaggtatagctgaactatggagtgttttgagtgtgttgacgagggaaaggggcgaaattcattctgtcacccagcaagaaacggcacacatcatctatcacccacgtctctatgcacagatacatttagtgaatgagcaggagggttgtcagtgtttccaagttgaaggcaatgctaattaacccataacacagacagctggtacttctgtgttagtgtcttaagtcacttataaagtctactggttagggacaaatgcaatcacatagcaggatgctgtaaacggaccaaacttcagtcattaatatactgcaacaacacgggaatagagcagc

SEQ ID NO: 9 - DNA, 11436 bp, Oreochromis niloticus
  source 1..11436: mol_type = unassigned DNA
ccataccttagcgacgggctatgctaggccccgcccacggctcagtgggcactaaagacatagcatcgagtgtacgctggactactgcagttggaaacgggctacaaagtggcgtcgctgtgcgcacaaacacgctgagacgatggaaaacacgcaaagccaggtaattcttgaatatcatttcccctcgagtttgtcttttttttttctctctttgtttgtttttggactttttcaaatatgtcgtttagctaggaaataagtggaagcaggcacgtgaaaaggaaaaaaaagttttgtcatctaagcagaaatgaaggcgcaagtctgttttggttaaaataaaaaggcagagttggctactatacttgaagcgttttcccgtgctttacgcgattcgtttagcttgtgtttccggaggtgacgctctgctgttttctccgatgaaggcccacctgcagctaattatattaataaagcctcagccacggttttgttcagttgtagtcaaaactaccaacattgtttccccccccttttttttttcaagtactagctgggaatgtcaagcttgtacccgattttggagttctattcaaatgctggcttcatggttttccattctacacaatagaaatgaacagaaatgtatccaagtgggcacattatctagatagttcaatttcattctgtttaaacatattaaatgccaacatatgctttaaataaataattgatattgacacatcaaatcagcaacagaatcatcaaagaaaaagacaggttgacattgatgtttgcaagtcatcaaccagcattttctgttcacctgtgtagcccactaatcacatcacttgattcattcattgggttttgcataaatggttctacactacatttattagtgctgtaattgtaataaagaaatgacaaatcacctatatgatacatttcctttcccacttaatagcatttcagaccatttcaatttaatgtataaaggtcaacgtgcacacttgctgggcaggcacagaggactgcagacgagctttaggcagattagctgatgtaaaaagagagttaaggtagaagaaggaagtctggcatctgcttgcaaaaagcataacaaacacacaatactaaaacaagaccagacatgtctgacagcaagagcctgtgtcagcctcagatgatcatttattagaaacactgactcaactaagcatggctggagcactccacatgatccttatgctgtcatctcaaacacaccagagaagcagatgagcgttatcagcctattgttaaaattgtttgtttgcaagatgctgccaatcagaaggttattttaaagtgagccaaaccgcttatttcagacagaatgaactcaagagctgctataataactcttgggatctaagagaataacgtccctttatttattaatttgattttttttttttcaaagaatgcaagtctataaattagatggaactagagctgggcgataaaacgataacgatatgtattgcgaaataactttttctcgatagaaaaatcaaactattgcgataggcctcatctctcttgtcctcttagagaaaaaaacaaaacagccaatcaaaattaagtagcgcagagccgaaccaatcacagtcgcagcgtcacgtcacgtgacttgttatgtacagcacaagcgccaaggcgcacgtgtatttgtttgggaagcagccagccgggctgcccaggtaatggaggaaatgagtttgccgactagagaaaaatcaaccgagagcgtgagcgaaggttaccaaagaaaaaacagatgatggttccaatgccggagagcttgtcgaacggaagggccacagaagttccgtagtgtgaaggtatttcggctatttcaagtctgacaaaaaacagagtagcgcgcactgtaaattgtgccgaaagcaagtctggaaatacaataaagcggtgcatgctcagtctctgactgaaagcgctgattcgtcattcggcttttgtcagactaaagtaactgttaaaactgtttgaaaagctaagctatacaacaaggagagattgagaatttccttttagttctcagtttatttgatattgacaaaagttagtcaattttgtctgttcttctgtaaaacgacctaagatttatttttagaattaaaattttgtttctaagtggaattgacaatttagtctgtttgttttttctgttttgaaacttaaactctttagcggctgccttttgtgtagtttgcaatatttgcctttatttatctgaaactgaagtctcatgttccttaagtacatctaccctgttgaacttattatgggaaataaatattttaattaaaacaagctgctgattatttcacattttacttgtgagcaacggcacatttaaatcttacaaatatagttatttggcttatatcgtgatatatatcgttatcgcctgaaatgaaaaaaacatatcgtgatatgaaaaaatcttatatcacccagctctagatggaacaaaccaagaataaaccataaataagaatataccagtggttgaatctacaacatgacaaagaagaccagttacccacacatgtgatgtagagtggtgagtaagagctttaaaacttgtaataaatcttagtgctccatgatggacagaatccagcctgtgtaggcattctgagcaggcatgcatataaatcacatcattgtaattaagtacagacgtaaaaagagggaaaaacgagtgttattcttgcctcacaggacaaacaggactttattctaaagttgcctgaactgtagtattacataaacgatttagatttttcatttgagaagatcatcagtttggtgttgtcaattttcagaacaagtttatgcacaggttatgctgtagtgtagtgaaagctgaaatagagcccggctatgagtaggtacaaagtagtacatcagcatatcaccagcataaaatgagaatgtgctgtagtgatattattactgacattgtttcgtatataaaacgaataagtgatccaagaacagaaccctggagcaatcctgttgttacataagccaagctagaaccaagtccttctacccatacaccccgagacctgcctgatggagaagtttagaaccattcagcttattatgatcagctgtgtcaaatgtcaaggctgcacagcattgttgttttcaactgaattgatgaaatcatttattacttttaagggtgctgcagttgtacagtaatttgtcctgaagcatcactgaaagtcagctcaagtcttatttacagtcaggaagtctttcattagtttccacaactttaacaagagttggtcatttagaaattggcccataattgtttaaaactgatggacccccaccttttaaacaaaggggggaaagcagatttcatatggttagaattaaattggtactcaaaagattagaaaagtaactgctggtgcaattaaaatcagtagctagctttaaaaggaggggctccaaattgtctgggtctgctggttttctagcatctaaaagtgaggcctttgtgctatgctgctggtcagatgctgccaacaatacctcattcatgcaaacacactcaaacctatatcagggaaacttggcaaatcgataaccagctttatgtgaccgcacatcgtaactgctgatttttgggtaggtgaatcggaaaaactgtattttggactcccttggtagtaaacattaagatgatggacttgtgctggttctattttgtttccacatcaggtgctgaaccttgaacgggtgcaggccctggaaatctggttgaaagcaaccaacacaaagctgactcaagttaatggccagaggaaatatggaggaccacctgagggtgagagaagctgcagcggaccgcttcaggatgccatattttttacctaatttacctaaatgttctgaaatcttcacagcagtagtgccaatactgcctattctcttcagcattaagaaatatggagttttttggctcagaggtcttcagcatttcaccactgtcaccaagtgccaataatgatatagatttctcaaaggtaaaagttaatcagctgcctttgtttgtaagatgcagctgtgaaaagacaaatctctaaaagctaaatgtgcaaatacttgatggcactgacctaaaactgaacccctgcttgatcttatttggagctgaaacaagaaaaagcccggcttgctcaagcgggtcacatgtcactctattgtaaagacttggacttgctttgtaaagtgcactaaggacttgttgtaaattttgcatcgcatgaacttgaattttaaaattgcataatcagccatcccatttcctctctgtttcatggttagtgtgggaaggtcccacaccgggaccgcgctgtgaagtcttcatcagccagatcccacgggacacgtatgaggacatccttattcccctgttcagctccattgggccactctgggagttccggctgatgatgaacttcagtgggcagaaccgcggctttgcgtatgccaaatatggctcagctgctatagctgttgaagccatacgacagctgcacggtcacatggtggagcctggctaccgcatcagtgtacggcggagcacagagaagcgacacctttgtattggaggtctgcctgcttccactagacaagaaggcatactgcaggtatagctgaactatggagtgttttgagtgtgttgacgagggaaaggggcgaaattcattctgtcacccagcaagaaacggcacacatcatctatcacccacgtctctatgcacagatacatttagtgaatgagcaggagggttgtcagtgtttccaagttgaaggcaatgctaattaacccataacacagacagctggtacttctgtgttagtgtcttaagtcacttataaagtctactggttagggacaaatgcaatcacatagcaggatgctgtaaacggaccaaacttcagtcattaatatactgcaacaacacgggaatagagcagctgtgatagaatacagcattaatggatcagtggtacagaagtggaggaagcaagaagaatgagttgagtaaagtttgacttatctgactgttttgtttcacttgatgcgccttatgtatgaaaaatacggtaggtcttaatagatgtctggcccttcagtcacatctggtattgaggttgtcacttaactgctgtgtgctcatgctttgcttctaggtgctgcgtatgctggtagagggggtggagagagtttccctgaaggccggacctggtatagagggggtatctgctactgttgctttctcatctcaccatgcagcttctatggctaagaaagtgctggtggaaggtaggggagctcttgttcatatagatgagtgacttaatattgatcatggtcctatttgtgtgccggcctgggtgtcacttggttataaaccagtttctggttgtgcactgttactccactctcatcatcacttggtatggagcacagaaaactaggtctgaagtcagcagtcattttatgcacaagtcagttcagttttcaattaaaatacaaattatagcatgaaaataagtaaaatggttatttgtgaagtggagctccccaaaagctaatcagcttgtaaacactagaaaatgtcctgcaaattcataattcccacattgcaaaattctcattaccagtgatgagaataatggcaatggcatgacatccctgagtctcaatttttgtgttaaaagtggggacttcagattatatagcagtctttaaattgtgttgatcagccatgtaaaaccagagttattataaaaaatatacgcaatatttttattttcctgaatactgttgttatgcctcatatgatgaggcggcacaatgccacggtggttagcactgttgctgcacagccagaaggtcctgagttcaattccagcatgaggccggggtctttctgtgtggagtttgcatgttctccccgtgtttccggcagttccctccgggtactccggcttcctcccaccgtccaaagacatgcagtttgtgggataggtcaattgattaatccaaattgcccataggtgtgaatgcgggagtgaatgtgagcgcgaatggttgtctgtctctgtgtgttagccctgcgacagactggcgacctgtccagggtgtaccccgcctctcgccctatgacagctgggataggctccagcgccccccgtttccattcggaagcgaatggaaaatgttatgttatgtttactgcaggaagaaacgttaaaaattgcgttttctaaggaaagcgcttgcaagcactcgctgcttgtgaccaaaaaagagaaggaaaaaacacccctttcctattggtggaaaaacgtaacatgtcgaccaatcaaaaaattatatgcaacatggcgtttggttgtttcggaagagggggaagtagggagagagatttgtgatgttcagcgtgtttggagagtgtagttggtgtgtagtataggtagttgttttgttttgtgtcagatcaatgaggcgactactccagccactgccaaaaagccaccaaagacagactgcagtataaacactgtctccaggtagaaaacgggaggagtgatacacctcctgctgtcaggcctgcaggtatcaggctgtgatgtgttccactgtcttatagtggacagaagttatttttattggagtggcacaaataatttctctggtatcttatttgatgcagaacagctgattgttctgtgaagagtttgtaatgttaaaaagaaagaaaaccaccttggctgaatttttaggtaaatagctgcatacaactttgttgtttgcataacttgtgcataggcttaaaaatttactttttcaattcagttcaattcaattttatttatatagcgccaaatcacaacagcagtcgcctcaaggcgctttatattgtacagtagatcctacaataatagatacagagaaaaacccaacaagttttaagttgtgaaaatgatccaataaacgtgtttgtggtcgttacagtaaaaaaatctaacttttttctacgctgattttatgttttttagtctgatttttagattaattgtgttaatacagtatgtcaaaatgggggaaaaaataactgtaaattcaaacaaggcgaggtaaacagtttttaaaggtgaaatgtagaggtcaaatcaaaagtagtcaaaaacagccaattataccctgaaccccagagggttcaactttttttttttttttttttttttttttttttaagtaacgcagtagttactttttctagtaattacttttatagtattgtaactcagtttttaaatcagttacttttatgaagaagtaactataactactttttcaaagtaacttggccaacactgcttattacattatattctaaatcactaaatatccatgtaagtccatatggagcggtgtccaaacataactcagttgtataaaatgctttgagtgctcagagttgtgagtccttcgaagctaaatttgtggacctgtacaggacgcagtaaatcttaagtctaaatcctgcattatacataacacaagctttatgaaatgaaaaaactttgtaacaatgacagagaggatcagaggagtatctaggtccctctgatgatatggctcttataacttcttttatttgtgaaggttgtaatctctaaagatacataggataagtattgcaccatataacattatgataatggtgactggtataaaataagcagtgtttcacatgcaagaaaatgtctcaatcaaaaatattaaaccaacataattatatatatttatgttcattgtcagtatgcatcttgaatttcagaaattcatcagtatgaagcatcaaaaataataattgatgacacattgctcaactatggcgtcaggaagaacaacagttttatgacccttatgaaagacctcgttgaatacctttgaacattgtttctgagcacccctagaagtttcaacaaatttagtgtaatatgcttacacgctcgtataatgtgagtcagcttatttttatgcttttatcattctttgtttcatcacttggagttttaatacttttaataagaacatacatgtagaaatagatttttaagatgctccctagtaagtgctctttttctttttttaaattataaactgaatctcatgttttagctttaagttttctagttggtgtcaataaatttgtttcattcagtattttcactttcatctattaagataagtgaatagtactatagcttaatggtctgtaatgtcaaaaagaattaggccactattgaaacaagtatgatgaatatttgtgatgttaacacaactatagtgtatgcatttatagtatacactactgtatgccaggagtgaacaagatgtcaactcactgatccataacactaggatctataatgaagactggagaagtgtagaaggtaaccatgcacacattcaggacagctacaagtatctggccaaaagaggagatggaagctaaaagacaaggaagctcctccagtccagcactccaagactgcatgctaagtgaaacaaaggtggctgaggactagtgcatgtcagaaccactatccagaatggggaaaaaaaacaaagatccataagttacatcaggaagatgttcagaactaaatacctcgggtagcagaaacccaagaaagaagaggaaccaccatgagaggacagatggaagacgtggttgatgtatggagaagtcctaccagtgctggacaaagctggaatgaaagacaccatagaggcactaattatggcagcactggaacaagctctaatcacaagttcaatctgcactggggtccactacaccaggcaaaccccaggtgcaggctgtgcagagatgccccaaaactatcacataagcacataacagcaggctgcaaaatgctagcagggtagacatggaacgccataaccaagtttctatacagaaacgtctgtgctgagtatgacctggaagtctcgaggtcaaaataggagatgcagccagggtggtggagaatgactgagctaagatcctgtgagacctccagatacagacggacaaactggtgattgctaaccaacaggacaccgtagtggtgaacaagcaaaaggaagacggcattaataatagataggcaagtgataacaacacagatactgttgttatcacttgcctgtttaacagagttgtgcaaagaaaagcgaactttgtagctgtttgttttacatggtgaaaatctacagaagctgcattaaaatatgcagtcttaactcgttctgatgtttcaattggctagcttgctaaaggctgatggcagtcttaccagtttctgtgacagtgaggactgcagcaagaagggaggatactaaaccatctttatctcaaggatttcataacattatttttcagatttaaagtgaaatggaatattaaagtgtgtagctgttgttgtttgggtttttttctcccacaggctaataaaatgtccatgggccattaaaactctgagccattcatcttgggctattaggttggacaatgaattatcatcattaattaataacaactgcaccagtgtcctgttttcagcagcaaaaacttttgcatcaaagccaaacagttttcattattgcagccaccatttaggcttaaaactgtacatactcatgcacaagtgactttaacatgcacacacaagtccacactcagtccaccttctctaggagataacatactttatgtaattaataaggagtagtattggggccacattaacaaaatcatacattgatctttttgagaataaagtagaaatctatagattaaagcccttgcataggtgagttagtaaaatcatacttcagaatcagttttggttacatggacatttctcttttaggacataacgtcagtgtggtgacaagcaagaatgttgacgtcatggtgcagaaagctgcatctgggcagaagatgaaaacacacaaacctggaagagtcggtcagcttggtgcaaatgaaatggcagggagtggacagatggaaggattacaccctcgatcaatgtgcttatacaataagacagctgatcaacttgtttcactaactgaaatacacatagtgtagtagctgtaggagccacggcagtcgtttggctttgagaaaaaaagaccaaatgtagagattacagttcaaaatactatttcaagtttaatgtcgaaattttgactttaaactcaaacacaaatttctttttttaatggtttggccagtggatcaaccaagagcctgatggggcttccagtggaaaccacttgaaattctaagtttgcatgtctatcttactattaacatttgacaattctccatttgaatcctttaaatgtgttaattctgctgtgttctgtctcataatgaatctctaagatttgatctgttgtttttttaaaactgtatttttggttgaaggagaaattcttcctgctaatctcacttcttcactgttttctcgtgtcttcagcatttaagaagcagtttgcaatgtgtgtgtcagtcaagtggcagccaacagagaagccaaaccctgacgagccacgatgccctcagaaacgtgcaaagagcctgttgccgtcacacctagggcccctgcaccacagttctccacaaccctcaggcccgccttcattcctgaccctccctgcatccatacccgcaggtttctgcagagcagtgggagggcccactgctcctcagctcgctcaccctacatgctcttttcccaattcctccacccaaggccatcttgtatttgcagcatccccagtgatgcttctcagtaagctgtgtgagagagcaggtgttggccagccgttttatgaagtgcagtacagccacgctgggcctgatggattcctctacttctcctataaggtgcagatccgcgggatcactgccgctttcaaggggttggtcatgatcttaccgggtcctaatgccagcaccatgctagaggaggctcagaaggctgtagcccagcaggtcctgcagaagatgtacaacactggtctcacacactaaacagctgatgccgtcctgcagttctgtttcaccttgtttgtgttatgtggtttcattttctgcatgtttttactagagtagcaccaagtttgtttctctgactataacttgtggtttgttttatgcatgatttttactgtacattagtgttctgtgttactggattggttctcattttaattaaatgagctttgaaaagaaa

SEQ ID NO: 10 - DNA, 23 bp, Oreochromis niloticus
  source 1..23: mol_type = other DNA
gatcttcggcctagactgcgagg

SEQ ID NO: 11 - DNA, 669 bp, Escherichia coli
  source 1..669: mol_type = other DNA
agcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggg

SEQ ID NO: 12 - DNA, 1044 bp, Escherichia coli
  source 1..1044: mol_type = other DNA
gtctgacgctcagtggaacgaaaactcacgttaagggattttggtcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttt

SEQ ID NO: 13 - DNA, 4151 bp, synthetic construct
  source 1..4151: mol_type = other DNA
gatcttcggcctagactgcgaggctcgagcttcacagcagtagtgccaatactgcctattctcttcagcattaagaaatatggagttttttggctcagaggtcttcagcatttcaccactgtcaccaagtgccaataatgatatagatttctcaaaggtaaaagttaatcagctgcctttgtttgtaagatgcagctgtgaaaagacaaatctctaaaagctaaatgtgcaaatacttgatggcactgacctaaaactgaacccctgcttgatcttatttggagctgaaacaagaaaaagcccggcttgctcaagcgggtcacatgtcactctattgtaaagacttggacttgctttgtaaagtgcactaaggacttgttgtaaattttgcatcgcatgaacttgaattttaaaattgcataatcagccatcccatttcctctctgtttcatggttagtgtgggaaggtcccacaccgggaccgcgctgtgaagtcttcatcagccagatcccacgggacacgtatgaggacatccttattcccctgttcagctccggatccggcccaagctacatcaagtgacctttttaaccaggtcagtgatttgtcatccattcgaaagctctggagagtctaagataaaacatacaataacccacacctcccccatagtcattatatagaaaagagaggggaaaaaaagtgccctgtcccactgcgggccccttttgtgctgttcctgccaacacagcagacctcctgctatatagaccctgctcccagccccacacactcaacagcaccatcccatccgacctgccaacaccagccaagcttcccatcgattcgaattcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgatatcagtcgacattgggccactctgggagttccggctgatgatgaacttcagtgggcagaaccgcggctttgcgtatgccaaatatggctcagctgctatagctgttgaagccatacgacagctgcacggtcacatggtggagcctggctaccgcatcagtgtacggcggagcacagagaagcgacacctttgtattggaggtctgcctgcttccactagacaagaaggcatactgcaggtatagctgaactatggagtgttttgagtgtgttgacgagggaaaggggcgaaattcattctgtcacccagcaagaaacggcacacatcatctatcacccacgtctctatgcacagatacatttagtgaatgagcaggagggttgtcagtgtttccaagttgaaggcaatgctaattaacccataacacagacagctggtacttctgtgttagtgtcttaagtcacttataaagtctactggttagggacaaatgcaatcacatagcaggatgctgtaaacggaccaaacttcagtcattaatatactgcaacaacacgggaatagagcagcactagtcctcgcagtctaggccgaagatcagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacgggcccgggaggcctgtctgacgctcagtggaacgaaaactcacgttaagggattttggtcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgagcggatacacgtagtg

SEQ ID NO: 14 - DNA, 4142 bp, synthetic construct
  source 1..4142: mol_type = other DNA
gatcttcggcctagactgcgaggctcgagcttcacagcagtagtgccaatactgcctattctcttcagcattaagaaatatggagttttttggctcagaggtcttcagcatttcaccactgtcaccaagtgccaataatgatatagatttctcaaaggtaaaagttaatcagctgcctttgtttgtaagatgcagctgtgaaaagacaaatctctaaaagctaaatgtgcaaatacttgatggcactgacctaaaactgaacccctgcttgatcttatttggagctgaaacaagaaaaagcccggcttgctcaagcgggtcacatgtcactctattgtaaagacttggacttgctttgtaaagtgcactaaggacttgttgtaaattttgcatcgcatgaacttgaattttaaaattgcataatcagccatcccatttcctctctgtttcatggttagtgtgggaaggtcccacaccgggaccgcgctgtgaagtcttcatcagccagatcccacgggacacgtatgaggacatccttattcccctgttcagctccggatccggcccaagctacatcaagtgacctttttaaccaggtcagtgatttgtcatccattcgaaagctctggagagtctaagataaaacatacaataacccacacctcccccatagtcattatatagaaaagagaggggaaaaaaagtgccctgtcccactgcgggccccttttgtgctgttcctgccaacacagcagacctcctgctatatagaccctgctcccagccccacacactcaacagcaccatcccatccgacctgccaacaccagccaagcttcccatcgattcgaattcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagtctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgatatcagtcgacattgggccactctgggagttccggctgatgatgaacttcagtgggcagaaccgcggctttgcgtatgccaaatatggctcagctgctatagctgttgaagccatacgacagctgcacggtcacatggtggagcctggctaccgcatcagtgtacggcggagcacagagaagcgacacctttgtattggaggtctgcctgcttccactagacaagaaggcatactgcaggtatagctgaactatggagtgttttgagtgtgttgacgagggaaaggggcgaaattcattctgtcacccagcaagaaacggcacacatcatctatcacccacgtctctatgcacagatacatttagtgaatgagcaggagggttgtcagtgtttccaagttgaaggcaatgctaattaacccataacacagacagctggtacttctgtgttagtgtcttaagtcacttataaagtctactggttagggacaaatgcaatcacatagcaggatgctgtaaacggaccaaacttcagtcattaatatactgcaacaacacgggaatagagcagcactagtcctcgcagtctaggccgaagatcagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacgggcccgggaggcctgtctgacgctcagtggaacgaaaactcacgttaagggattttggtcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgagcggatacacgtagtg

SEQ ID NO: 15 - DNA, 326 bp, Oreochromis niloticus
  source 1..326: mol_type = genomic DNA
tgtgggaaggtcccacaccgggaccgcgctgtgaagtcttcatcagccagatcccacgggacacgtatgaggacatccttattcccctgttcagctccattgggccactctgggagttccggctgatgatgaacttcagtgggcagaaccgcggctttgcgtatgccaaatatggctcagctgctatagctgttgaagccatacgacagctgcacggtcacatggtggagcctggctaccgcatcagtgtacggcggagcacagagaagcgacacctttgtattggaggtctgcctgcttccactagacaagaaggcatactgcag

SEQ ID NO: 16 - DNA, 23 bp, Oreochromis niloticus
  source 1..23: mol_type = other DNA
ttcccctgttcagctccattggg

SEQ ID NO: 17 - DNA, 22 bp, synthetic construct
  source 1..22: mol_type = other DNA
gtttccacatcaggtgctgaac

SEQ ID NO: 18 - DNA, 20 bp, synthetic construct
  source 1..20: mol_type = other DNA
cggatgggatggtgctgttg

SEQ ID NO: 19 - DNA, 22 bp, synthetic construct
  source 1..22: mol_type = other DNA
gtccaggagcgcaccatcttct

SEQ ID NO: 20 - DNA, 24 bp, synthetic construct
  source 1..24: mol_type = other DNA
gcagttaagtgacaacctcaatac

SEQ ID NO: 21 - DNA, 21 bp, synthetic construct
  source 1..21: mol_type = other DNA
ccacctacaaggccaagaagc

SEQ ID NO: 22 - DNA, 575 bp, Takifugu alboplumbeus
  source 1..575: mol_type = genomic DNA
ggccatggtggaaagtggagctgcagctggagaaattaacttatagagtgacacgtaaaaatcagaacattgttgttgtgatggagtaggactgacaactgctgtaccccatttgtgggccaaaacatgcaaggggtacatcaatatgctagtcggagctttgttctttctctcctgttattttctatgttcttttagattctgaaccttgaccgagtgaaggcatttcacgagtggctggaaagaaccaacacaaagataactcaagtcaatggccagagaaagtatggaggaccacctgacagtgagagaggctctatgtaccattactgggtgaaatctggtggatggttttataccataagatgatttaaataacgtgtttctgagtagaatcctaattaatgtagtaccatgtattttagtgagaccatgtgaattagtgtagcaaatattcaaaggttcacatctgtattctaccaccagagtgggacgggccagttccaggagcccggtgtgaaatctacatcactcatattccacgggatacctacgaggacgtgctgattcctctg

SEQ ID NO: 23 - DNA, 545 bp, Takifugu alboplumbeus
  source 1..545: mol_type = genomic DNA
ttcaccactgttgggccactctggcagtttcggcttatgatgaacttcagtggtcagaatcgcggctttgcctatgccaaatatggctcaccagccatcgctgccagtgccattcaactgctgcatggtcacatgatcgagcccggtgtctctctgtgtgttcgccatagcacagagaagcgacagctgtgcatcggaggcttgccagtgagcaccaagcaagctgacatttctcaggtacgacacgtttgataaaaggcgcctgtttcctctttttaatatttaatagttccattagatttgttggaaaaagttttgttctgcagtaatcaaattggtatttaccagtccctctccagtatctgaagtcctggaacatggagatgtgtgattttcttctcccacgctgtggataatatctttttgctaagaagaaaaattgacttctttgtgtctttaactttgcacctgtagcatcttcctctggtgttttttttttttattctaaaaggatttaataagcgcctgttgcgtggcaagtatgc

SEQ ID NO: 24 - DNA, 8625 bp, Takifugu alboplumbeus
  source 1..8625: mol_type = genomic DNA
cactgagctcctgagaacggcccattcttttacaattatttgtagaaacagtctccatgcccaagggcttgattttatacacctgtggctgggccaagtgattaggacacctgattctgatcatttggatggtggtcaaatacttttggcaatatagtgtagctgttgttgctcagttttacccgttgttttgtcgcagctgccatttatgaatgtaagtcatctcttagtgtgttctcttctgtcttgttactgtcatgaatttcttgcatgtgcagcaggttggtggaaatgttaacacctccaatggatgatttgatgtttgagcgttgccaccacttaatcatatttttggtttgtgagtgtgaaatttgtgcttcccatgaatgccactactgtgagatgtgtgaacaagtgagtggcactggacgaggtgtccagtcttatcgcattttggatgtgtaggaagaaaaatattcgatgctaaggtcaacagcaaagcttaggcttaattggtccacagctcaggccttgtgtgtatgaatatggttttagatacgattccacatatattttatccaatataactgcacatacagtataatcaccataactgagttctacgtaagcatatgatgatgtgatgtaaccaccataactgagttccatataagcatatgctgaagagttttaagtgcagctcggacggaaaaactttccctgcttcattttggaagcacagctcatcatgcggaagttttaagagggcaaacggagcataagagagcgaagaagggaataagagcgcgaacaggagcataaggcgtcatctcagctcttagaattttacaacctcacactgagagtaccctggggttggtgttcttcctcttttgcttttttaataattcctagcataccccccacttttgggttgatttaaaactagtttatgggtgtcaaataaccctcaatcaattttatatttagcagaatactctctcttactaccaatgtacttccacttaaccttcacttggtggtattgaatgagcagagtttagaaaaggtttctgctttgcctttttgctgtcttggctccagttgggtgagagtccaccagatagagaaagtgcgctgtggctgggttgatgagtctctcctgaggccattttccacgagtagagtctttttctggtttctctacctcaggggtggggaacccccggcctccgggccgtatacggcccgcgagaccatttctaccggcccgcaacgcaataagatttttatattttatatgtgcccttatacagtgggaccgttcgctaaactccgcgagctgcgagtagcagcagtagcgtatttttcccaaaccatcactgacccactaccaaaccggtcatgctgaatgatgttgagggcagcataacgttctccacggcatctccagacccttttacatctgacgcatgtgctcaggttgaacctgctctcatcggtgaagagcacagggtgccagtggcgtagttgccaattctggtggtctatggtctggggaggcatatccctggaaggacgcacagataatggcaccctgacttctattcggtatcgggatgaaatctttggagccattgtcagaacctacgctggtgcagtgagacctgggttccacctggtccacgacactgcctgacctcatgtggctagagtatgcaggcagttcctggaggatgaagtaattgataccattgactggcccccacgttcacctgacctaaacccaatagaacacctctgggacattatgtttaggtccatccggtctgattaggagcatgccccgacgctgtcaggcatgcgtgcaagcacgtgggggccacacaaaccactgagaatcattttgagttgctacaataacattttggcaaaatagaccagtctgctgcattatttttttcattttttggggcgtctttgaattcccccctctatagggtgatctttttaatttctatccaattaggtagcatcattttgttactaatacatcacctactttttatcagggcagatattcaagataattccccccgtttagatctgatgtgttcctctaatttttttgagcagtgtatattctaggtcttacattttattatgtattttactgtaccttttgtgttttttcttaaatgttatgtacatatattattctgtatactttaatactaataataatactatttatttagtaaagcagaaataaacgtgattttcagtcaataaatcaaaggctgcttcagcaggcactaatctctgaaggtagctgtgactaggccccttttggaagacgggtgaactgaatggttactgaatggttctgtctaaactaactataggccactttcgaatctttttatatccaagattactctaaaattaattcctaaatgactcagcagagtcaggcatttgacctaaccacagaacagaaacagttcttgttagactattaaatgagattagggtgaacagtggtaaggtgtgagttttggttttattggatctaataacagcatgtgacactacatactataacattcagatacacatgctttaaacttgggcaagactcagtggaatcatttttctcttgaaaagaattatgtcttacttgagggagtagtcattttgcgaccactagagattatgaatctgaccaaatgcatgtgtggagtcccgcaggggtcagactttaggtcttctctgttcagtttaactaatgttggttatcacagttacacagatgacacacaaatttatctagcacttttcctagacaaactgggtcttatgagtctctgtgtcagtgttgagagcaaattaacaattgcatgaaccaacattgtctgctaaccatatgttaacaaaacagattgctgtatttggaaataaaatgagaaaatgattcctgtcctgagcaaaaaccttgagtcatttgcccccaaaaccaaagaccaagtcctaaagcaaggaatgttttcttgagttaaatttgaccgttgacatatcaaattattaactaaaaatcaacgcatgcagagtgaagggttttatgttgcaaaactcatatccatgctttcagttagagtaatctagcctactgtctagtcaacaagtttatttttgggccccttgacgatattgcacattgtttgctcatttaatcactctaaagtacttcattattttaaattgcactccagcatttgacaaaatatattttatgaaggtttttgttatacgatgtaacttttaatgtattaatatttaaaacaaataaagcacaaataaaactcccttgccgtgcctacagggggcagaattatcgctcaaaaatcttaattgaggcaccgaattgcgtcgacaatggcgcctgcagacgacgtttcgtcgtggccgtgtcaagtgtgacctgttagccaattgggttcgtcagttgaaggcgctgctccctcatttccaagaaaggcgggcatgatggatgaatcccagagccaggtaaactgatcagaaatgcgaaatatcgccagtcgctttttttttttgccgacgtctgtatttttttcaacaaaacgaaatgaaagcaaacagtaaaactacaacaaagagttgtcacgcgttctaaaaagcggcaaaactttgatgtgccttagatgtgctagtttgtttgaaaccattttcgtgattttagcgccaaagtgtaggttttgttttacgttgaatgcaccgaccaacgtagacggacattggatgccttttaaaaaaataaataaaaaacaaaacaaaaaaacgtgacattctttcagttacgtgaaattggctaaacgcgtaaaatctgcgcaggaaaacgttagcgatgtgcggtattatttttgtaagcatttattatcacaagaatgattataaacgataggtctaaaacttaacaaaaaaagttaagcttttgttatgtatacttaattttgttccgaagcaatttaatgcaatggtgcactatgactcgaaggataaaagtgattatttgattttagattgtcatctatacaactatggtattttaatatgtcattaatttaaaatctctcttaatatgttgctccttctcatttttgtcagagcggacatatttctaaacacgagtgcctttttaatttttctgttttaattgcttaatatattattttggccattggtagaagcaatatttaaacttgtctccatattttagtgatagtgtaatgtatgatccatcattaacagacaattgtgtgcaggtgtttactgtagcattaaaatccaaacttttgagtgttttcatggttgaggatactttattaggtacaccttgctaataaaaattggaccctttttgccttcagaactgccttagttcttcttgtcatactgtaaataagaggttggaaacattcctcagcaatcttggtccatattgacaagaccaaataatgcctgacctaattcctacagcgttgtcagctgcacatttatgaattaaatctcccatttgttcactttccaaaggtgctctattttattgagatttggggagtgtgagggcccttggactactgtgaattcattgccatgttacagaaactagattgaggtgatttgagctttgagatatggtgtattctgatgctggaagtagccatcaaaataaaatggtccactgtggtcataaagggatgagcatggtcagcaacaatactcaagtaggatttggtgtttgaaagacaccagggcttaaaggagccaagactggtattgcaattgcaatcaaaatacaggtacaagacagatagatacaaaagccagcttcagtaagaacgtctccatttgaccaatgttcttctgttaggttttatattgccataaagaccgtgcaaatgttgggagattttcctcagtgtctcagcattgtcacagcaaagtttatactattttattaagtattaagattaaatatctaatatctcatattagaaaacgttgttataggatgaaggaggaggcacctgcagcaatgtgtttggaggccaaaaacgaatcgttgcatatagaaaatgagatcctgagtatgtgtgaatcatggtcacgtttttaatgatttattgtacaccttcagtaattacattaattaagattatggtaatcaccctttgctctgtcttacatgtcaaggagaaaattagcaactttcgtgccttcgaattcttttctactttcagccgtctccagttttcaaaggcatctcttatacagtatagatccttgatttgtttctgacagaagtcagaaacaaagataggtggatggtttaagaacagcccaaaacacaccaaaaatacatttttgtaaacatatttactggctgacaactttgcaatattatggctggagtagtgttgtaaaattttaagttaacgtgattccttattgtcttaatgttggcatatcagggctattttaagactgttttctttaggtattttggttttgtgtcagatttccagaatgcagagattgactgcaatgaaaaacctgatgaataaaaacacatatgtgtaagggattcttcacatgacaataacaacggcaagttttcatggcatttgcaaatctcatatacacaagagcagtgcccagagtgcccgggctccagagtagatttttgacattgttatctttatttctttgcgtaaacatgaagtgcagttttcagttaaatcagtgatgtgcactgcattcgctttaatctctggtagcacgcaacttggaaaagcaacaacagctaacgccggctaccaagcaacatgtttgctgctaacagtactaggtttgtttttactaagttgttttgctctaacaaaatgtggatttgctgcattaaactcagacaattaaattgccagtggagagtggagcgtatttgttcgccaaggcctcattcagtcgcctagattgccacctgacatgcacgctactgagttttctgtggttgatgtttttatgtctccatgtgaacgcggatcgctttggaaacaaaggaaaaacctttgtgtttgttaccttaggttgtatattggcttcactcttgaatataaagatcaaatgataaaataccaacaacacgtgtacctaataaactggccagggactcttttccataggtttttggccatggtggaaagtggagctgcagctggagaaattaacttatagagtgacacgtaaaaatcagaacattgttgttgtgatggagtaggactgacaactgctgtaccccatttgtgggccaaaacatgcaaggggtacatcaatatgctagtcggagctttgttctttctctcctgttattttctatgttcttttagattctgaaccttgaccgagtgaaggcatttcacgagtggctggaaagaaccaacacaaagataactcaagtcaatggccagagaaagtatggaggaccacctgacagtgagagaggctctatgtaccattactgggtgaaatctggtggatggttttataccataagatgatttaaataacgtgtttctgagtagaatcctaattaatgtagtaccatgtattttagtgagaccatgtgaattagtgtagcaaatattcaaaggttcacatctgtattctaccaccagagtgggacgggccagttccaggagcccggtgtgaaatctacatcactcatattccacgggatacctacgaggacgtgctgattcctctgttcaccactgttgggccactctggcagtttcggcttatgatgaacttcagtggtcagaatcgcggctttgcctatgccaaatatggctcaccagccatcgctgccagtgccattcaactgctgcatggtcacatgatcgagcccggtgtctctctgtgtgttcgccatagcacagagaagcgacagctgtgcatcggaggcttgccagtgagcaccaagcaagctgacatttctcaggtacgacacgtttgataaaaggcgcctgtttcctctttttaatatttaatagttccattagatttgttggaaaaagttttgttctgcagtaatcaaattggtatttaccagtccctctccagtatctgaagtcctggaacatggagatgtgtgattttcttctcccacgctgtggataatatctttttgctaagaagaaaaattgacttctttgtgtctttaactttgcacctgtagcatcttcctctggtgttttttttttttattctaaaaggatttaataagcgcctgttgcgtggcaagtatgcttaaaaagcacctggaatatttctgtctgttcagtttttttggattaatctcagagatttgaaaactgacatctggattttttccccctctaaattgcatactgctgtcctgatcttgtaggtgatgcacaccatgtgtgagggaatagagagagtttctctaaaatcagagcctggtattgctggaatgtctgctgtagttaccttttcttcccattacacagcatcaatggcaaagaaagtggtgattgaaggtatgtgagatgctaaactgatgcttggaatgtatgagtgaattactgttaatgtagttaacgtttatctgaatagatttacttttttcccccaagcattcaagaaccagtatgccttgagggtctcagtctcctggttctcatcctttaaggccagtccaaaccggttactgcctccacaaaatcttgtaaatgtctcttgtcggtcctctgtcttgcttcctcgccagaccggcacgttattccactcccctagtttttgcaaagcagtcggaggacctatcaccccacagccttatgaatgttccaccccctcctctcgtgcgtctcttccatcccccatgatgctcctcaataacttgtgccagacagctggttttggcccgccacactataagatttcctgcagccgtgcaggacctgatgggtacctccatttttcttatcaagtgtttattcttgggataaatatgagccttgaaggggtgatcatgatcttacctggtgccaacatctccagcactatggaggaggctcgggaggctgtagcacagcaggtcttgaggaaggtcttccaccgccaggtctcccactgagcaggcctgcacagttcagcaacttctactgcaccgctcaatgctgttttatttcatagagttgttcaaaaaacattgatatgtatattttattgcagttaacttatttttgcatggttttattagtatttgctgttgtttgttctgttctatgcatgcttgttgttgttgtgctcagttaatcattttaagtaaatgtgacttcaaaagtaaatctgatgtgttggattctttggggttgtaaagtgattgtttatacataaaagaataatcaatcgtcacagtgaaactcagtccggatcagaatctcagcttgtccagtttagaagcaatgatctcagaacagtacccaaattaaggttcgctctggagaaaggcatccgcta

SEQ ID NO: 25 - DNA, 4184 bp, synthetic construct
  source 1..4184: mol_type = other DNA
gatcttcggcctagactgcgaggctcgagggccatggtggaaagtggagctgcagctggagaaattaacttatagagtgacacgtaaaaatcagaacattgttgttgtgatggagtaggactgacaactgctgtaccccatttgtgggccaaaacatgcaaggggtacatcaatatgctagtcggagctttgttctttctctcctgttattttctatgttcttttagattctgaaccttgaccgagtgaaggcatttcacgagtggctggaaagaaccaacacaaagataactcaagtcaatggccagagaaagtatggaggaccacctgacagtgagagaggctctatgtaccattactgggtgaaatctggtggatggttttataccataagatgatttaaataacgtgtttctgagtagaatcctaattaatgtagtaccatgtattttagtgagaccatgtgaattagtgtagcaaatattcaaaggttcacatctgtattctaccaccagagtgggacgggccagttccaggagcccggtgtgaaatctacatcactcatattccacgggatacctacgaggacgtgctgattcctctgggatccggcccaagctacatcaagtgacctttttaaccaggtcagtgatttgtcatccattcgaaagctctggagagtctaagataaaacatacaataacccacacctcccccatagtcattatatagaaaagagaggggaaaaaaagtgccctgtcccactgcgggccccttttgtgctgttcctgccaacacagcagacctcctgctatatagaccctgctcccagccccacacactcaacagcaccatcccatccgacctgccaacaccagccaagcttcccatcgattcgaattcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgatatcagtcgacttcaccactgttgggccactctggcagtttcggcttatgatgaacttcagtggtcagaatcgcggctttgcctatgccaaatatggctcaccagccatcgctgccagtgccattcaactgctgcatggtcacatgatcgagcccggtgtctctctgtgtgttcgccatagcacagagaagcgacagctgtgcatcggaggcttgccagtgagcaccaagcaagctgacatttctcaggtacgacacgtttgataaaaggcgcctgtttcctctttttaatatttaatagttccattagatttgttggaaaaagttttgttctgcagtaatcaaattggtatttaccagtccctctccagtatctgaagtcctggaacatggagatgtgtgattttcttctcccacgctgtggataatatctttttgctaagaagaaaaattgacttctttgtgtctttaactttgcacctgtagcatcttcctctggtgttttttttttttattctaaaaggatttaataagcgcctgttgcgtggcaagtatgcactagtcctcgcagtctaggccgaagatcagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacgggcccgggaggcctgtctgacgctcagtggaacgaaaactcacgttaagggattttggtcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgagcggatacacgtagtg

SEQ ID NO: 26 - DNA, 4175 bp, synthetic construct
  source 1..4175: mol_type = other DNA
gatcttcggcctagactgcgaggctcgagggccatggtggaaagtggagctgcagctggagaaattaacttatagagtgacacgtaaaaatcagaacattgttgttgtgatggagtaggactgacaactgctgtaccccatttgtgggccaaaacatgcaaggggtacatcaatatgctagtcggagctttgttctttctctcctgttattttctatgttcttttagattctgaaccttgaccgagtgaaggcatttcacgagtggctggaaagaaccaacacaaagataactcaagtcaatggccagagaaagtatggaggaccacctgacagtgagagaggctctatgtaccattactgggtgaaatctggtggatggttttataccataagatgatttaaataacgtgtttctgagtagaatcctaattaatgtagtaccatgtattttagtgagaccatgtgaattagtgtagcaaatattcaaaggttcacatctgtattctaccaccagagtgggacgggccagttccaggagcccggtgtgaaatctacatcactcatattccacgggatacctacgaggacgtgctgattcctctgggatccggcccaagctacatcaagtgacctttttaaccaggtcagtgatttgtcatccattcgaaagctctggagagtctaagataaaacatacaataacccacacctcccccatagtcattatatagaaaagagaggggaaaaaaagtgccctgtcccactgcgggccccttttgtgctgttcctgccaacacagcagacctcctgctatatagaccctgctcccagccccacacactcaacagcaccatcccatccgacctgccaacaccagccaagcttcccatcgattcgaattcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagtctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgatatcagtcgacttcaccactgttgggccactctggcagtttcggcttatgatgaacttcagtggtcagaatcgcggctttgcctatgccaaatatggctcaccagccatcgctgccagtgccattcaactgctgcatggtcacatgatcgagcccggtgtctctctgtgtgttcgccatagcacagagaagcgacagctgtgcatcggaggcttgccagtgagcaccaagcaagctgacatttctcaggtacgacacgtttgataaaaggcgcctgtttcctctttttaatatttaatagttccattagatttgttggaaaaagttttgttctgcagtaatcaaattggtatttaccagtccctctccagtatctgaagtcctggaacatggagatgtgtgattttcttctcccacgctgtggataatatctttttgctaagaagaaaaattgacttctttgtgtctttaactttgcacctgtagcatcttcctctggtgttttttttttttattctaaaaggatttaataagcgcctgttgcgtggcaagtatgcactagtcctcgcagtctaggccgaagatcagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacgggcccgggaggcctgtctgacgctcagtggaacgaaaactcacgttaagggattttggtcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgagcggatacacgtagtg

SEQ ID NO: 27 - DNA, 326 bp, Takifugu alboplumbeus
  source 1..326: mol_type = genomic DNA
agtgggacgggccagttccaggagcccggtgtgaaatctacatcactcatattccacgggatacctacgaggacgtgctgattcctctgttcaccactgttgggccactctggcagtttcggcttatgatgaacttcagtggtcagaatcgcggctttgcctatgccaaatatggctcaccagccatcgctgccagtgccattcaactgctgcatggtcacatgatcgagcccggtgtctctctgtgtgttcgccatagcacagagaagcgacagctgtgcatcggaggcttgccagtgagcaccaagcaagctgacatttctcag

SEQ ID NO: 28 - DNA, 23 bp, Takifugu alboplumbeus
  source 1..23: mol_type = other DNA
ggcccaacagtggtgaacagagg

SEQ ID NO: 29 - DNA, 20 bp, synthetic construct
  source 1..20: mol_type = other DNA
ccacctgacatgcacgctac

SEQ ID NO: 30 - DNA, 23 bp, synthetic construct
  source 1..23: mol_type = other DNA
acattgatgagtttggacaaacc

SEQ ID NO: 31 - DNA, 23 bp, synthetic construct
  source 1..23: mol_type = other DNA
cttcaatcaccactttctttgcc